(19) United States
(12) Patent Application Publication
(10) Pub. No.: US 2015/0080673 A1
(43) Pub. Date: Mar. 19, 2015

(54) BLOOD PRESSURE ESTIMATION APPARATUS AND METHOD

(71) Applicants: Shinya Hashimoto, Kanagawa (JP); Kiyoshi Tateishi, Kanagawa (JP); Atsuya Ito, Kanagawa (JP); Hiroyuki Ishihara, Kanagawa (JP); Masaki Goma, Kanagawa (JP)

(72) Inventors: Shinya Hashimoto, Kanagawa (JP); Kiyoshi Tateishi, Kanagawa (JP); Atsuya Ito, Kanagawa (JP); Hiroyuki Ishihara, Kanagawa (JP); Masaki Goma, Kanagawa (JP)

(73) Assignee: PIONEER CORPORATION, Kanagawa (JP)

(21) Appl. No.: 14/394,921

(22) PCT Filed: Apr. 20, 2012
(86) PCT No.: PCT/JP2012/060768

§ 371 Date: Nov. 17, 2014

(51) Int. Cl. A61B 5/021; A61B 5/00; A61B 5/026

(52) U.S. Cl. CPC A61B 5/021; A61B 5/026; A61B 5/7278; A61B 5/7221
USPC 600/301

(57) ABSTRACT

A blood pressure estimation apparatus (1) is provided with: a blood pressure measuring device (11) which measures a blood pressure (BPm) of a living body every first period; a blood flow measuring device (12) which measures a blood flow volume (BF) of the living body every second period which is shorter than the first period; and a blood pressure estimating device (13) which estimates the blood pressure (BPc) every third period which is shorter than the first period, on the basis of the blood pressure which is measured by the blood pressure measuring device and the blood flow volume which is measured by the blood flow measuring device.

### TECHNICAL FIELD

The present invention relates to a blood pressure estimation apparatus and method which estimate a blood pressure of a living body (especially, which sequentially estimate the blood pressure), for example.

### BACKGROUND ART

As this type of a blood pressure apparatus, for example, an apparatus which converts a sequential change of an area of a blood vessel, which is obtained from a reflected wave of an ultrasonic wave (a reflected ultrasonic wave) with which a living body is irradiated, into a sequential blood pressure value as a physiology parameter is proposed as disclosed in a Patent Literature 1.

Moreover, as this type of a blood apparatus, an apparatus which calculates a correspondence curve and sequentially estimates a blood pressure by using the correspondence curve, wherein the correspondence curve represents a correspondence relation between a power of a sound of a blood flow which is obtained from the sound of the blood flow and an estimated blood pressure is proposed as disclosed in a Patent Literature 2.

Moreover, as this type of a blood apparatus, an apparatus which monitors a change of the blood pressure by using a blood pressure measuring unit and a pulse moving time measuring unit is proposed as disclosed in a Patent Literature 3.

Moreover, as this type of a blood pressure estimation apparatus, an apparatus which extracts a pulse wave signal by detecting a light with which the living body is irradiated and which calculates the change of the blood pressure from a temporal change of a second order differential value of the pulse wave signal is proposed as disclosed in a Patent Literature 4.

### CITATION LIST

### Patent Literature

Patent Literature 1: Japanese Patent Application Laid Open No. Hei11-309144

Patent Literature 2: International Publication No. 2009/125811

Patent Literature 3: Japanese Patent Application Laid Open No. 2009-528860

Patent Literature 4: Japanese Patent Application Laid Open No. 2011-167424

### SUMMARY OF INVENTION

### Technical Problem

It is an object of the present invention to provide, for example, a blood pressure estimation apparatus and method which are capable of appropriately estimating the blood pressure by using a method which is different from the methods disclosed in the Patent Literature 1 to the Patent Literature 4 as described above.

### Solution to Problem

A blood pressure estimation apparatus which is to solve the above described technical problem is provided with: a blood pressure measuring device which measures a blood pressure of a living body every first period; a blood flow measuring device which measures a blood flow volume of the living body every second period which is shorter than the first period; and a blood pressure estimating device which estimates the blood pressure every third period which is shorter than the first period, on the basis of the blood pressure which is measured by the blood pressure measuring device and the blood flow volume which is measured by the blood flow measuring device.

A blood pressure estimation method which is to solve the above described technical problem is provided with: a blood pressure measuring process which measures a blood pressure of a living body every first period; a blood flow measuring process which measures a blood flow volume of the living body every second period which is shorter than the first period; and a blood pressure estimating process which estimates the blood pressure every third period which is shorter than the first period, on the basis of the blood pressure which is measured by the blood pressure measuring process and the blood flow volume which is measured by the blood flow measuring process.

### BRIEF DESCRIPTION OF DRAWINGS

FIG. 1 is a block diagram illustrating a configuration of a blood pressure estimation apparatus in a first example.

FIG. 2 is a flowchart illustrating the flow of the operation of the blood pressure estimation apparatus in the first example.

FIG. 3 is a graph illustrating a blood pressure and a blood flow volume which are measured by the blood pressure estimation apparatus in the first example.

FIG. 4 is a graph illustrating a blood pressure and a blood flow volume which are related to an operation of the blood pressure estimation apparatus which estimates a blood pressure at a desired time point on the basis of a blood flow volume at a time point which is prior to a reference time point and a blood flow volume at a time point which is prior to the desired time point.

FIG. 5 is a graph illustrating a blood pressure and a blood flow volume which are related to an operation of the blood pressure estimation apparatus which estimates a blood pressure at a desired time point on the basis of an average value of a blood flow volume within a predetermined time span defined by using a reference time point as a base and an average value of a blood flow volume within a predetermined time span defined by using the desired time point as a base.

FIG. 6 is a flowchart illustrating the flow of the operation of the blood pressure estimation apparatus in the first example which adjusts a weighting coefficient as occasion demands.

FIG. 7 is a block diagram illustrating a configuration of a blood pressure estimation apparatus in a second example.

FIG. 8 is a flowchart illustrating the flow of the operation of the blood pressure estimation apparatus in the second example.

FIG. 9 is a flowchart illustrating the flow of another operation of the blood pressure estimation apparatus in the second example.

FIG. 10 is a graph illustrating a blood pressure and a blood flow volume which are related to another operation of the blood pressure estimation apparatus in the second example.

FIG. 11 is a block diagram illustrating a configuration of a blood pressure estimation apparatus in a third example.

FIG. 12 is a flowchart illustrating the flow of the operation of the blood pressure estimation apparatus in the third example.

FIG. 13 is a block diagram illustrating a configuration of a blood pressure estimation apparatus in a fourth example.

FIG. 14 is a flowchart illustrating the flow of the operation of the blood pressure estimation apparatus in the fourth example.

### DESCRIPTION OF EMBODIMENT

Hereinafter, as an embodiment of the present invention, an embodiments of a blood pressure estimation apparatus and method will be described.

(Embodiment of Blood Pressure Estimation Apparatus)

<1>

A blood pressure estimation apparatus of the present embodiment is provided with: a blood pressure measuring device which measures a blood pressure of a living body every first period; a blood flow measuring device which measures a blood flow volume of the living body every second period which is shorter than the first period; and a blood pressure estimating device which estimates the blood pressure every third period which is shorter than the first period, on the basis of the blood pressure which is measured by the blood pressure measuring device and the blood flow volume which is measured by the blood flow measuring device.

According to the blood pressure estimation apparatus of the present embodiments, the blood pressure measuring device measures the blood pressure of the living body. The blood pressure measuring device measures the blood pressure of every first period. In other words, the blood pressure measuring device measures the blood pressure every first period. More specifically, the blood pressure measuring device measures the blood pressure at a first timing. Then, the blood pressure measuring device measures the blood pressure once again at a second timing which is obtained by an elapse of a time span corresponding to the first period from the first timing. After that, the blood pressure measuring device repeats a same operation.

The blood flow measuring device measures the blood flow volume of the living body. The blood flow measuring device measures the blood flow volume of every second period. In other words, the blood flow measuring device measures the blood flow volume every second period. More specifically, the blood flow measuring device measures the blood flow volume at a third timing. Then, the blood flow measuring device measures the blood flow volume once again at a fourth timing which is obtained by an elapse of a time span corresponding to the second period from the third timing. After that, the blood flow measuring device repeats a same operation.

Here, the second period in which the blood flow measuring device measures the blood flow volume is shorter than the first period in which the blood pressure measuring device measures the blood pressure. Namely, a frequency at which the blood flow measuring device measures the blood flow volume is higher than a frequency at which the blood pressure measuring device measures the blood pressure. In other words, the number of times which the blood flow measuring device measures the blood flow volume within a certain time span is larger than the number of times which the blood pressure measuring device measures the blood pressure within the certain time span.

The blood pressure estimating device estimates the blood pressure of every third period on the basis of the blood pressure which is measured by the blood pressure measuring device (namely, the blood pressure of every first period) and the blood flow volume which is measured by the blood flow measuring device (namely, the blood flow volume of every second period). In other words, the blood pressure estimating device estimates the blood pressure every third period on the basis of the blood pressure which is measured by the blood pressure measuring device every first period and the blood flow volume which is measured by the blood flow measuring device every second period.

Here, the third period in which the blood pressure estimating device estimates the blood pressure is shorter than the first period in which the blood pressure measuring device measures the blood pressure. Namely, a frequency at which the blood pressure estimating device estimates the blood pressure is higher than a frequency at which the blood pressure measuring device measures the blood pressure. In other words, the number of times which the blood pressure estimating device estimates the blood pressure within a certain time span is larger than the number of times which the blood pressure measuring device measures the blood pressure within the certain time span. Therefore, the blood pressure estimating device is capable of estimating the blood pressure at a time point when the blood pressure measuring device does not measure the blood pressure, for example. Incidentally, as described later in detail, the third period in which the blood pressure estimating device estimates the blood pressure may be same as the second period in which the blood flow measuring device measures the blood flow volume.

As described above, the blood pressure estimation apparatus of the present embodiment is capable of estimating the blood pressure of every third period (namely, the blood pressure which is relatively sequential) on the basis of the blood pressure which is measured by the blood pressure measuring device (namely, the blood pressure of every first period and the blood pressure which is relatively discrete) and the blood flow volume which is measured by the blood flow measuring device (namely, the blood flow volume of every second period and the blood flow volume which is relatively sequential). In other words, the blood pressure estimation apparatus of the present embodiment is capable of sequentially estimating the blood pressure on the basis of the blood pressure which is discretely measured by the blood pressure measuring device and the blood flow volume which is sequentially measured by the blood flow measuring device. Moreover, in other words, the blood pressure estimation apparatus of the present embodiment is capable of estimating the blood pressure every third period (namely, relatively sequentially estimating the blood pressure) on the basis of the blood pressure which is measured by the blood pressure measuring device every first period and the blood flow volume which is measured by the blood flow measuring device every second period.

Incidentally, all apparatuses which are disclosed in the above described Patent Literature 1 to the above described Patent Literature 4 do not mention the method of estimating the sequential blood pressure by using the sequential blood flow volume at all. However, the blood pressure estimation apparatus of the present embodiment is capable of estimating the sequential blood pressure by using the sequential blood flow volume which a Laser Doppler blood flow meter and so on is capable of measuring relatively easily. Therefore, the blood pressure estimation apparatus of the present embodiment is capable of estimating the sequential blood pressure relatively easily.

Incidentally, the “first period” in the present embodiment may be a constant value or may be a variable value. Namely, the “first period” in the present embodiment mainly represents a cycle of each operation when same operation (for example, the measurement of the blood pressure) is repeatedly performed periodically or non-periodically.

Same argument can be applied to the “second period” and the “third period”.

<2>

In another aspect of the blood pressure estimation apparatus of the present embodiment, the blood pressure estimating device estimates the blood pressure at a desired time point on the basis of the blood pressure at a reference time point which is measured by the blood pressure measuring device and a ratio or a change rate of the blood flow volume at the desired time point which is measured by the blood flow measuring device to the blood flow volume at the reference time point which is measured by the blood flow measuring device

According to this aspect, the blood pressure estimating device is capable of estimating the blood pressure at the desired time point on the basis of the blood pressure at the reference time point and the ratio or the change rate of the blood flow volume at the desired time point to the blood flow volume at the reference time point. The reason is as follows. There is often a certain correlation between the temporal change of the blood pressure and the temporal change of the blood flow volume. Thus, the blood pressure likely changes in a manner which is same as a manner in which the blood flow volume changes. Therefore, according to this aspect, the blood pressure estimating device is capable of estimating the blood pressure appropriately.

For example, the blood pressure estimating device may regard a value which is obtained by multiplying the blood pressure at the reference time point by the ratio of the blood flow volume at the desired time point to the blood flow volume at the reference time point as the blood pressure at the desired time point. Alternatively, the blood pressure estimating device may regard a value which is obtained by adding a multiplied value to the blood pressure at the reference time point as the blood pressure at the desired time point, wherein the multiplied value is obtained by multiplying the blood pressure at the reference time point by the change rate of the blood flow volume at the desired time point to the blood flow volume at the reference time point.

<3>

In another aspect of the blood pressure estimation apparatus which estimates the blood pressure at the desired time point on the basis of the blood pressure at the reference time point and the ratio or the change rate of the blood flow volume at the desired time point to the blood flow volume at the reference time point as described above, the blood pressure estimating device estimates the blood pressure at the desired time point on the basis of the ratio or the change rate which is weighted depending on a predetermined reflecting coefficient.

According to this aspect, the blood pressure estimating device is capable of estimating the blood pressure at the desired time point on the basis of not only the blood pressure at the reference time point and the ratio or the change rate of the blood flow volume at the desired time point to the blood flow volume at the reference time point but also the reflecting coefficient which represents a degree of the weighting to the ratio or the change rate. This is why the correlation between the temporal change of the blood pressure and the temporal change of the blood flow volume may vary depending on the individual difference of the living body. Thus, the blood pressure estimating device is capable of appropriately estimating the blood pressure while considering the individual difference of the living body (for example, absorbing or compensating the individual difference) by adjusting the reflecting coefficient to absorb or compensate the individual difference.

Moreover, the reflecting coefficient, which is one example of an information which is capable of specifying the individual difference, may be stored as an archival record (for example, as a database which represents the archival record) by a storing device every time the blood pressure measuring device measures the blood pressure or every time the blood pressure estimating device estimates the blood pressure, for example. Especially, considering that the reflecting coefficient is the information which is capable of specifying the individual difference of the living body, the reflecting coefficient may be stored in such a manner that the reflecting coefficient can be distinguished for each living body (for example, in such a manner that the reflecting coefficient is associated with the living body). In this case, the blood pressure estimating device is capable of improving an accuracy of estimating the blood pressure by using the reflecting coefficient which is stored in the storing device in estimating the blood pressure next time.

Incidentally, it is preferable that the storing of the reflecting coefficient by the storing device be performed especially when the reflecting coefficient is corrected (namely, the reflecting coefficient is updated as occasion demands) as described later. However, the storing of the reflecting coefficient by the storing device may be performed when the reflecting coefficient is not corrected (namely, the reflecting coefficient has a constant value). In this case, the storing device may stores the reflecting coefficient which is the constant value (namely, the reflecting coefficient in default).

<4>

In another aspect of the blood pressure estimation apparatus which estimates the blood pressure at the desired time point on the basis of the weighted ratio or change rate as described above, the blood pressure estimation apparatus is further provided with: a storing device which stores the blood pressure which is estimated by the blood pressure estimating device within a predetermined estimating time span, wherein the blood pressure estimating device estimates the blood pressure within the estimating time span, and then collectively corrects the blood pressure within the estimating time span ex-post facto by collectively weighting the blood pressure which is stored in the storing device and which is estimated within the estimating time span on the basis of the reflecting coefficient.

According to this aspect, the blood pressure estimating device is capable of collectively performing the weighting (namely, the weighting which influences the blood pressure which is already estimated in the past) based on the reflecting coefficient span ex-post facto on a series of the blood pressure which is estimated within the estimating time, in addition to or instead of performing the weighting (namely, the weighting which influences the estimation of the current blood pressure or the blood pressure in the future) based on the reflecting coefficient every time the blood pressure is estimated.

<5>

In another aspect of the blood pressure estimation apparatus which estimates the blood pressure at the desired time point on the basis of the weighted ratio or change rate as described above, the blood pressure estimating device corrects the reflecting coefficient such that a difference between the blood pressure at the reference time point which is measured by the blood pressure measuring device and the blood pressure at the reference time point which is estimated by the blood pressure estimating device decreases.

According to this aspect, the blood pressure estimating device is capable of correcting the reflecting coefficient which is used for the weighting such that the difference between the blood pressure which is measured by the blood pressure measuring device and the blood pressure which is estimated by the blood pressure estimating device is absorbed or compensated. For example, the blood pressure estimating device may correct the reflecting coefficient such that the difference (especially, an absolute value of the difference) between the blood pressure which is measured by the blood pressure measuring device and the blood pressure which is estimated by the blood pressure estimating device becomes zero. As a result, the blood pressure estimating device is capable of estimating the blood pressure appropriately.

<6>

In another aspect of the blood pressure estimation apparatus which corrects the reflecting coefficient as described above, the blood pressure estimation apparatus is further provided with: a storing device which stores the reflecting coefficient which is corrected by the blood pressure estimating device, wherein the blood pressure estimating device estimates the blood pressure at the desired time point on the basis of the ratio or the change rate which is weighted depending on the reflecting coefficient which is stored in the storing device.

According to this aspect, the reflecting coefficient, which is one example of the information which is capable of specifying the individual difference, may be stored as the archival record (for example, as the database which represents the archival record) by the storing device every time the blood pressure measuring device measures the blood pressure or every time the blood pressure estimating device estimates the blood pressure, for example. In this case, the blood pressure estimating device is capable of improving the accuracy of estimating the blood pressure by using the reflecting coefficient which is stored in the storing device in estimating the blood pressure next time.

Incidentally, as described above, considering that the reflecting coefficient is the information which is capable of specifying the individual difference of the living body, the reflecting coefficient may be stored in such a manner that the reflecting coefficient can be distinguished for each living body (for example, in such a manner that the reflecting coefficient is associated with the living body). In addition, in this aspect, the reflecting coefficient is likely corrected as time goes on. Therefore, the reflecting coefficient may be stored in such a manner that the reflecting coefficient can be distinguished for each time (for example, in such a manner that the reflecting coefficient is associated with the time).

<7>

In another aspect of the blood pressure estimation apparatus of the present embodiment, the blood pressure estimation apparatus is further provided with: a storing device which stores the blood pressure which is estimated by the blood pressure estimating device within a predetermined estimating time span, wherein the blood pressure estimating device estimates the blood pressure within the estimating time span, and then collectively corrects the blood pressure within the estimating time span ex-post facto by collectively performing a predetermined filtering process on the blood pressure which is stored in the storing device and which is estimated within the estimating time span.

According to this aspect, the blood pressure estimating device is capable of performing the predetermined filtering process (namely, the filtering process which influences the blood pressure which is already estimated in the past) on a series of the blood pressure which is estimated within the estimating time span correctively or ex-post facto.

<8>

In another aspect of the blood pressure estimation apparatus of the present embodiment, the blood pressure estimating device estimates the blood pressure at a desired time point on the basis of the blood pressure at a reference time point which is measured by the blood pressure measuring device and a ratio or a change rate of the blood flow volume at a time point prior to the desired time point by a predetermined time which is measured by the blood flow measuring device to the blood flow volume at a time point prior to the reference time point by a predetermined time which is measured by the blood flow measuring device.

According to this aspect, the blood pressure estimating device is capable of estimating the blood pressure at the reference time point on the basis of the ratio or the change rate of the blood flow volume at the time point prior to the desired time point by the predetermined time to the blood flow volume at the time point prior to the reference time point by the predetermined time, instead of the ratio or the change rate of the blood flow volume at the desired time point to the blood flow volume at the reference time point. This is why the correlation between the temporal change of the blood pressure and the temporal change of the blood flow volume may vary depending on the individual difference of the living body. More specifically, this is why a time which is required for the change of the blood flow volume to result in the change of the blood pressure may vary depending on the individual difference of the living body. Thus, according to this aspect, the blood pressure estimating device is capable of appropriately estimating the blood pressure while considering the individual difference of the living body (for example, absorbing or compensating the individual difference).

<9>

In another aspect of the blood pressure apparatus of the present embodiment, the blood pressure estimating device estimates the blood pressure at a desired time point on the basis of the blood pressure at a reference time point which is measured by the blood pressure measuring device and a ratio or a change rate of an average value of the blood flow volume within a predetermined time span defined by using the desired time point as a base which is measured by the blood flow measuring device to an average value of the blood flow volume within a predetermined time span defined by using the reference time point as a base which is measured by the blood flow measuring device.

According to this aspect, the blood pressure estimating device is capable of estimating the blood pressure at the reference time point on the basis of the ratio or the change rate of the average value of the blood flow volume within the predetermined time span defined by using the desired time point as a base to the average value of the blood flow volume within the predetermined time span defined by using the reference time point as a base, instead of the ratio or the change rate of the blood flow volume at the desired time point to the blood flow volume at the reference time point. Thus, the blood pressure estimating device is capable of appropriately estimating the blood pressure while eliminating an influence of a pulsation and a fluctuation caused by the living body included in the blood flow.

<10>

In another aspect of the blood pressure estimation apparatus of the present embodiment, the blood pressure estimating device (i) estimates the blood pressure if the blood pressure which is measured by the blood pressure measuring device satisfies a predetermined condition, and (ii) does not estimate the blood pressure if the blood pressure which is measured by the blood pressure measuring device does not satisfy the predetermined condition.

According to this aspect, the blood pressure estimating device is capable of estimating the blood pressure if the blood pressure satisfies the predetermined condition (for example, if it is preferable that the blood pressure be estimated). In other words, the blood pressure estimating device does not need to estimate the blood pressure if the blood pressure does not satisfy the predetermined condition (for example, if there is no need to estimate the blood pressure). Therefore, a power consumption of the blood pressure estimation apparatus decreases, in comparison with a blood pressure estimation apparatus in a comparative example which always keeps estimating the blood pressure.

<11>

In another aspect of the blood pressure estimation apparatus of the present embodiment, the blood flow measuring device (i) measures the blood flow volume if the blood pressure which is measured by the blood pressure measuring device satisfies a predetermined condition, and (ii) does not measure the blood flow volume if the blood pressure which is measured by the blood pressure measuring device does not satisfy the predetermined condition.

According to this aspect, the blood flow measuring device is capable of measuring the blood flow volume if the blood pressure satisfies the predetermined condition (for example, if it is preferable that the blood pressure be estimated). In other words, the blood flow measuring device does not need to measure the blood flow volume if the blood pressure does not satisfy the predetermined condition (for example, if there is no need to estimate the blood pressure). Therefore, a power consumption of the blood pressure estimation apparatus decreases, in comparison with a blood pressure estimation apparatus in a comparative example which always keeps measuring the blood flow volume.

<12>

In another aspect of the blood pressure estimation apparatus of the present embodiment, the blood pressure estimation apparatus is further provided with: a timer device which sets a timing at which the blood pressure measuring device measures the blood pressure, wherein the blood pressure measuring device automatically measures the blood pressure every first period by measuring blood pressure at the timing which is set by the timer device

According to this aspect, the blood pressure measuring device is capable of automatically measuring the blood pressure every first period. Incidentally, the blood pressure measuring device is likely provided with a blood pressure meter which needs a manual operation of an operator (for example, a non-invasive blood pressure meter which loops a cuff around an arm and applies a pressure to the arm via the cuff), for example. Even in this case, the blood pressure measuring device is capable of automatically measuring the blood pressure every first period depending on the timing which the timer device sets, even when the operator does not match the timing (alternatively, the operator does not perform the manual operation).

<13>

In another aspect of the blood pressure estimation apparatus which is provided with the timer device as described above, the timer device sets the timing such that (i) a frequency of the timing at which the blood pressure is measured when the blood pressure which is measured by the blood pressure measuring device satisfies a predetermined condition is higher than (ii) a frequency of the timing at which the blood pressure is measured when the blood pressure which is measured by the blood pressure measuring device does not satisfy the predetermined condition.

According to this aspect, the timer device is capable of appropriately setting the timing, at which the blood pressure measuring device measures the blood pressure, depending on the blood pressure which is measured by the blood pressure measuring device. For example, the timer device is capable of setting the timing at which the blood pressure measuring device measures the blood pressure such that the blood pressure is measured at a relatively high frequency if the blood pressure satisfies the predetermined condition (for example, if it is preferable that the blood pressure be estimated). On the other hand, the timer device is capable of setting the timing at which the blood pressure measuring device measures the blood pressure such that the blood pressure is measured at a relatively low frequency if the blood pressure does not satisfy the predetermined condition (for example, if there is no need to estimate the blood pressure).

(Embodiment of Blood Pressure Estimation Method)

<14>

A blood pressure estimation method of the present embodiment is provided with: a blood pressure measuring process which measures a blood pressure of a living body every first period; a blood flow measuring process which measures a blood flow volume of the living body every second period which is shorter than the first period; and a blood pressure estimating process which estimates the blood pressure every third period which is shorter than the first period, on the basis of the blood pressure which is measured by the blood pressure measuring process and the blood flow volume which is measured by the blood flow measuring process.

According to the blood pressure estimation method of the present embodiment, it is possible to enjoy various effects which the above described blood pressure estimation apparatus of the present embodiment enjoys.

Incidentally, the blood pressure estimation method of the present embodiment may adopt various aspects in accordance with the various aspect which the blood pressure estimation apparatus of the present embodiment adopts.

These operations and other advantages of the present embodiment will become more apparent from the examples explained below.

As described above, the blood pressure estimation apparatus of the present embodiment is provided with the blood pressure measuring device, the blood flow measuring device and the blood pressure estimating device. The blood pressure estimation method of the present embodiment is provided with the blood pressure measuring process, the blood flow measuring process and the blood pressure estimating process. Therefore, it is possible to estimate the blood pressure appropriately.

### EXAMPLES

Hereinafter, examples of the blood pressure estimation apparatus will be described with reference to the drawings.

### (1) First Example

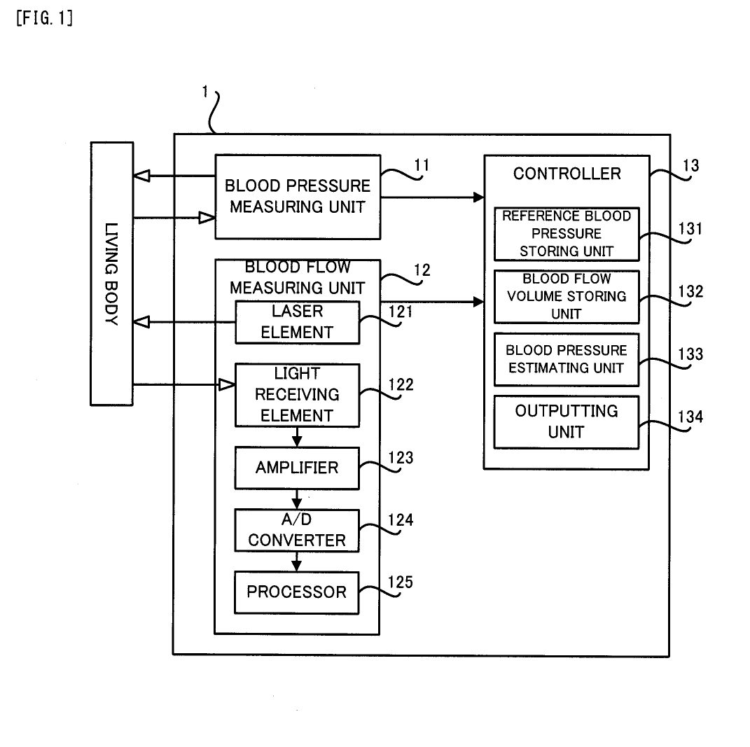

Firstly, with reference to FIG. 1 to FIG. 6, a blood pressure estimation apparatus 1 in a first example will be explained.

### (1-1) Configuration of Blood Pressure Estimation Apparatus

Firstly, with reference to FIG. 1, a configuration of the blood pressure estimation apparatus 1 in the first example will be explained. FIG. 1 is a block diagram illustrating the configuration of the blood pressure estimation apparatus 1 in the first example.

As illustrated in FIG. 1, the blood pressure estimation apparatus 1 in the first example is provided with: a blood pressure measuring unit 11; a blood flow measuring unit 12; and a controller 13.

The blood pressure measuring unit 11 measures a blood pressure BPm (n: incidentally, n is a variable number representing a time point) of a living body (for example, a human, an animal and the like), for example. The blood pressure measuring unit 11 may be a non-invasive blood pressure meter (for example, a blood pressure meter which measures the blood pressure BPm(n) by looping a cuff around an arm and applying a pressure to the arm via the cuff). However, the configuration of the blood pressure measuring unit 11 may be arbitrary as long as the blood pressure measuring unit 11 is capable of measuring the blood pressure BPm(n) by using any method.

The blood flow measuring unit 12 measures a blood flow volume (namely, a flow volume of a blood which flows in a blood vessel) BF(n) of the living body. A Laser Doppler blood flow meter may be used as the above described blood flow measuring unit 12, for example. However, the configuration of the blood flow measuring unit 12 may be arbitrary as long as the blood flow measuring unit 12 is capable of measuring the blood flow volume BF (n) by using any method. Hereinafter, the explanation will be provided by using an example in which the blood flow measuring unit 12 is the Laser Doppler blood flow meter, for the purpose of the explanation.

The blood flow measuring unit 12 is provided with: a laser element 121; a light receiving element 122; an amplifier 123; an A/D (Analogue to Digital) converter 124; and a processor 125.

The laser element 11 irradiates the living body with a laser light.

In this case, it is preferable that the laser element 11 irradiate the blood vessel in the living body with the laser light. Especially, it is preferable that the laser element 11 irradiate the blood vessel of an ear lobe with the laser light. However, the laser element 11 may irradiate the blood vessel of another portion with the laser light.

The light receiving element 122 receives a beat signal light which is generated by a mutual interference between a reflected light from the living body and a scattered light of the laser light from the living body. The light receiving element 12 generates a detection electric current which is obtained by converting the received beat signal light into an electric signal.

The amplifier 123 converts the detection electric current which is outputted from the light receiving element 122 into a voltage signal and amplifies the voltage signal.

The A/D converter 124 performs an A/D converting process (namely, a quantizing process) on the output of the amplifier 123 (namely, the voltage signal which depends on the beat signal light which is received by the light receiving element 122). As a result, the A/D converter 124 outputs a sample value of the voltage signal (namely, the quantized voltage signal), which depends on the beat signal light which is received by the light receiving element 122, into the processor 125.

The processor 125 performs a frequency analysis on the output of the A/D converter 124 (namely, the sample value of the voltage signal which depends on the beat signal light which is received by the light receiving element 122) by using a FFT (Fast Fourier Transform). As a result, the processor 125 calculates the blood flow volume BF(n).

The controller 13 is a central controlling device (for example, a CPU: Central Processing Unit) for controlling the blood pressure estimation apparatus 1. The controller 13 is provided with: a reference blood pressure storing unit 131; a blood flow volume storing unit 132; a blood pressure estimating unit 133; and an outputting unit 134, as processing circuits which are physically realized therein or processing blocks which are logically realized therein.

The reference blood pressure storing unit 131 is a memory which stores a reference blood pressure BP(s), wherein the reference blood pressure BP(s) is used when the blood pressure estimating unit 133 estimates the blood pressure BPc(n). Incidentally, the reference blood pressure BP(s) is a blood pressure BPm(n) which is measured by the blood pressure measuring unit 11, for example. Especially, the reference blood pressure BP(s) may be the latest blood pressure BPm(n) among the blood pressures BPm(n) which are measured by the blood pressure measuring unit 11, for example. More specifically, the reference blood pressure BP(s) may be the blood pressure BPm(s) which is measured at the latest time point (a reference time point s) among the blood pressures BPm(n) which are measured by the blood pressure measuring unit 11, for example.

The blood flow volume storing unit 132 is a memory which stores the blood flow volumes BF(n) which are measured by the blood flow measuring unit 12. Incidentally, it is preferable that the blood flow volume storing unit 132 store the blood flow volumes BF(n) which are measured by the blood flow measuring unit 12 within a certain time span. Alternatively, the blood flow volume storing unit 132 may store all of the blood flow volumes BF(n) which are measured by the blood flow measuring unit 12.

Incidentally, the reference blood pressure storing unit 131 may be physically separated from the blood flow volume storing unit 132. Alternatively, single memory may constitute the reference blood pressure storing unit 131 and the blood flow volume storing unit 132.

The blood pressure estimating unit 133 estimates the blood pressure BPc(n) on the basis of the reference blood pressure BP(s) which is stored in the reference blood pressure storing unit 131 and the blood flow volume BF(n) which is stored in the blood flow volume storing unit 132. For example, the blood pressure estimating unit 133 estimates the blood pressure BPc(t) at a desired time point t. Especially, the blood pressure estimating unit 133 is capable of estimating the blood pressure BPc(t) at the desired time point t at which the blood pressure measuring unit 11 does not measure the blood pressure BPm(n). In other words, the blood pressure estimating unit 133 is capable of estimating the blood pressure BPc(n) every period which is shorter than a period every which the blood pressure measuring unit 11 measures the blood pressure BPm(n). Moreover, in other words, the blood pressure estimating unit 133 is capable of sequentially estimating the blood pressure BPc(n) of the living body on the basis of the blood pressures BPm(n) which are discretely measured by the blood pressure measuring unit 11.

The outputting unit 134 outputs the blood pressure BPc(n) which is estimated by the blood pressure estimating unit 133 into an instrument which is outside of the blood pressure estimation apparatus 1. For example, the outputting unit 134 sequentially outputs the blood pressure BPc(n), which is sequentially estimated by the blood pressure estimating unit 134, into the instrument which is outside of the blood pressure estimation apparatus 1.

Incidentally, hereinafter, the blood pressure BP(n) which is measured by the blood pressure measuring unit 11 is referred to as the “blood pressure BPm(n)” and the blood pressure BP(n) which is estimated by the blood pressure estimating unit 133 is referred to as the “blood pressure BPc(n)”, and thus both are distinguished, for the purpose of the explanation. However, if both do not need to be distinguished, the explanation will be provided by simply calling the “BP(n)”

### (1-2) Operation of Blood Pressure Estimation Apparatus

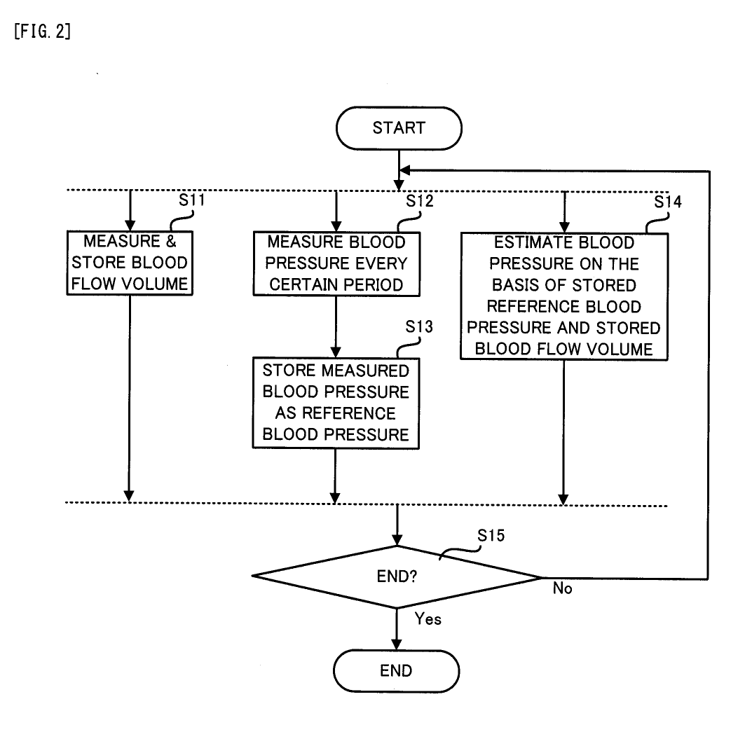

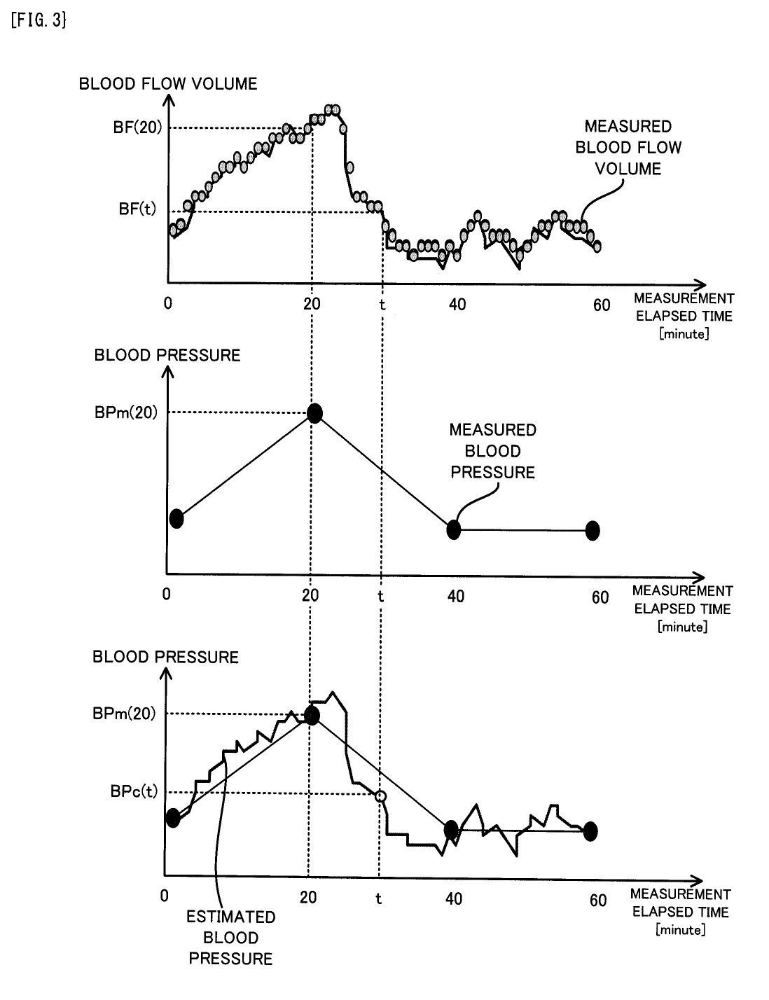

Next, with reference to FIG. 2 to FIG. 3, a flow of an operation of the blood pressure estimation apparatus 1 in the first example will be explained. FIG. 2 is a flowchart illustrating the flow of the operation of the blood pressure estimation apparatus 1 in the first example. FIG. 3 is a graph illustrating the blood pressure BP(n) and the blood flow volume BF(n) which are measured by the blood pressure estimation apparatus 1 in the first example.

As illustrated in FIG. 2, the blood flow measuring unit 12 measures the blood flow volume BF(n) of the living body (step S11). Incidentally, the measurement of the blood flow volume BF(n) of the living body by the blood flow measuring unit 12 is performed continuously until the estimating operation of the blood pressure BP(n) by the blood pressure estimation apparatus 1 is ended (step S15).

Specifically, the laser element 121 irradiates the living body with the laser light.

Then, the light receiving element 122 receives the beat signal light which is generated by the mutual interference of the scattered lights of the laser light from the living body (more specifically, the mutual interference between a scattered light which is scattered by a blood cell which is a moving scattering substance and a scattered light which is scattered by a static tissue). Specifically, when the living body is irradiated with the laser light, the scattered light which is caused by a flow of the blood (namely, the movement of the red blood cell which is the scattering substance) in the blood vessel of the living body is generated. The frequency of the scattered light is different from the frequency of the original laser light, due to a Laser Doppler function depending on a moving velocity of the blood. The light receiving element 122 receives the beat signal light (what we call a frequency differential signal) which is generated by the mutual interference of the above described scattered lights. Incidentally, a forward scattered light which corresponds to a transmitted light of the laser light LB with which the living body is irradiated may be used as the scattered light which generates the beat signal light.

Then, the light receiving element 122 generates the detection electric current which is obtained by converting the received beat signal light into the electric signal. The light receiving element 122 outputs the generated detection electric current into the amplifier 123. The amplifier 123 converts the detection electric current (namely, the detection electric current which depends on the beat signal light which is received by the light receiving element 122) which is outputted from the light receiving element 122 into the voltage signal and amplifies the voltage signal. The amplifier 123 outputs the voltage signal into the A/D converter 124.

Then, the A/D converter 124 performs the A/D converting process (namely, the quantizing process) on the output of the amplifier 123 (namely, the voltage signal which depends on the beat signal light which is received by the light receiving element 122). As a result, the A/D converter 124 outputs the sample value of the voltage signal (namely, the quantized voltage signal), which depends on the beat signal light which is received by the light receiving element 122, into the processor 125. Specifically, if a sampling cycle of the A/D converter 124 is Ta, the A/D converter 124 outputs the sample value of the voltage signal (namely, the quantized voltage signal), which depends on the beat signal light which is received by the light receiving signal 122, every cycle Ta.

Then, the processor 125 performs the frequency analysis on the output of the A/D converter 124 (namely, the sample value of the voltage signal which depends on the beat signal light which is received by the light receiving signal 12) by using the FFT (Fast Fourier Transform). As a result, the processor 125 calculates the blood flow volume BF(n). Specifically, for example, the processor 125 performs the FFT on the sample value of the voltage signal which depends on the beat signal light. The processor 125 calculates the blood flow volume BF(n) by using a primary (first) moment which is a result of a multiplication of a power spectrum and a frequency vector which are obtained by performing the FFT. Incidentally, as a method of calculating the blood flow volume BF(n) by performing the frequency analysis using the FFT, a well-known method (for example, Japanese Patent No. 3 313 841) may be used. Thus, its detailed explanation will be omitted. The processor 125 outputs the calculated blood flow volume BF(n) into the controller 13 (especially, the blood flow volume storing unit 132). As a result, the blood flow volume storing unit 132 stores the blood flow volumes BF(n) which are measured by the blood flow measuring unit 12.

Subsequent to, in tandem with or in parallel with the measurement of the blood flow volume BF(n) at the step S11, the blood pressure measuring unit 11 measures the blood pressure BPm(n) of the living body (step S12). Incidentally, the measurement of the blood pressure BPm(n) of the living body by the blood pressure measuring unit 11 is performed continuously until the estimating operation of the blood pressure BPc(n) by the blood pressure estimation apparatus 1 is ended (step S15).

Especially, the blood pressure measuring unit 11 measures the blood pressure BPm(n) every certain period (for example, every 20 minutes). For example, the blood pressure BPm(n) is measured every certain period by that an operator manipulates the blood pressure measuring unit 11 (for example, makes the cuff be loop around an arm of the living body and makes the cuff apply the pressure to the arm) every certain period.

Here, the period every which the blood pressure measuring unit 11 measures the blood pressure BPm(n) is longer than the period every which the blood flow measuring unit 12 measures the blood flow volume BF(n). For example, the blood pressure measuring unit 11 may measure the blood pressure BPm(n) every 20 minutes and the blood flow measuring unit 12 may measure the blood flow volume BF(n) every period which is shorter than 20 minutes (for example, every several dozen mili-seconds to several dozen seconds). Incidentally, if the blood pressure measuring unit 11 is the non-invasive blood pressure meter and the blood flow measuring unit 12 is the Laser Doppler blood flow meter, the period every which the blood pressure measuring unit 11 measures the blood pressure BPm(n) is likely longer than the period every which the blood flow measuring unit 12 measures the blood flow volume BF(n), because of a time consuming of measuring the blood pressure BPm(n).

Then, the blood pressure BPm(n) which is measured by the blood pressure measuring unit 11 is outputted to the controller 13 (especially, the reference blood pressure storing unit 131). As a result, the reference blood pressure storing unit 131 stores the blood pressure BPm(n) which is measured by the blood pressure unit 11 as the reference blood pressure BP(s) (step S13). Incidentally, in the first example, it is preferable that the reference blood pressure storing unit 131 store the newly measured blood pressure BPm(n) as the reference blood pressure BP(s) every time the blood pressure measuring unit 11 newly measures the blood pressure BPm(n). However, the reference blood pressure storing unit 131 may not store the newly measured blood pressure BPm(n) as the reference blood pressure BP(s) even when the blood pressure measuring unit 11 newly measures the blood pressure BPm(n). In other words, the reference blood pressure storing unit 131 may continue to store the previously stored reference blood pressure BP(s) without change even when the blood pressure measuring unit 11 newly measures the blood pressure BPm(n).

Subsequent to, in tandem with or in parallel with the measurement of the blood flow volume BF(n) at the step S11 and the measurement of the blood pressure BPm(n) at the step S12, the blood pressure estimating unit 133 estimates the blood pressure BPc(n) (step S14). For example, the blood pressure estimating unit 133 estimates the blood pressure BPc(t) at the desired time point t. Incidentally, the estimation of the blood pressure BPc(n) by the blood pressure estimating unit 133 is performed continuously until the estimating operation of the blood pressure BPc(n) by the blood pressure estimation apparatus 1 is ended (step S15).

For example, the blood pressure estimating unit 133 estimates the blood pressure BPc(t) at the desired time point t on the basis of the reference blood pressure BP(s) at a reference time point s which is stored in the reference blood pressure storing unit 131 and the blood flow volume BF(s) at the reference time point s and the blood flow volume (t) at the desired time point t which are stored in the blood flow volume storing unit 132.

In this case, the blood pressure estimating unit 133 may estimate the blood pressure BPc(n) at the time point at which the blood flow volume BF(n) is measured, every time the blood flow measuring unit 12 measures the blood flow volume BF(n). Namely, the blood pressure estimating unit 133 may estimate the blood pressure BPc(n) every period which is same as a period every which the blood flow measuring unit 12 measures the blood flow volume BF(n). Alternatively, the blood pressure estimating unit 133 may estimate the blood pressure BPc(n) every period which is different from the period every which the blood flow measuring unit 12 measures the blood flow volume BF(n). However, it is preferable that the blood pressure estimating unit 133 estimate the blood pressure BPc(n) every period which is shorter than a period every which the blood pressure measuring unit 11 measures the blood pressure BPm(n). In other words, it is preferable that the blood pressure estimating unit 133 estimate the blood pressure BPc(n) at a frequency which is higher than a frequency at which the blood pressure measuring unit 11 measures the blood pressure BPm(n).

Specifically, the blood pressure estimating unit 133 may regard a value which is obtained by multiplying a ratio A of the blood flow volume BF(t) at the desired time point t to the blood flow volume BF(s) at the reference time point s with the reference blood pressure BP(s) at the reference time point s as the blood pressure BPc(t) at the desired time point t. Namely, the blood pressure estimating unit 133 may estimate the blood pressure BPc(t) at the desired time point t by using a formula of BPc(t)=BP(s)*A=BP (s)*(BF(t)/BF(s)). For example, if the blood pressure BPm(20) which is measured by the blood pressure measuring unit 11 at the time point of “n=20 minutes” is the reference blood pressure BP(s), the blood pressure BPc(t) at the desired time point t is BPm(20)*(BF(t)/BF(20)).

Alternatively, the blood pressure estimating unit 133 may regard a value which is obtained by adding a multiplied value to the reference blood pressure BP(s) at the reference time point s as the blood pressure BPc(t) at the desired time point t, wherein the multiplied value is obtained by multiplying a change rate B of the blood flow volume BF(t) at the desired time point t to the blood flow volume BF(s) at the reference time point s with the reference blood pressure BP(s) at the reference time point s. Namely, the blood pressure estimating unit 133 may estimate the blood pressure BPc(t) at the desired time point t by using a formula of BPc(t)=BP(s)+BP(s)*B=BP (s)+BP(s)*((BF(t)−BF(s))/BF(s)). For example, if the blood pressure BPm(20) which is measured by the blood pressure measuring unit 11 at the time point of “n=20 minutes” is the reference blood pressure BP(s), the blood pressure BPc(t) at the desired time point t is BPm(20)+BPm(20)*((BF(t)−BF(20)/BF(20)).

Incidentally, in the above described explanation, the formula of BPc(t)=BP (s)*(BF(t)/BF(s)) and the formula of BPc(t)=BP(s)+BP(s)*((BF(t)−BF(s))/BF(s)) are listed as the examples. However, these two formulas are substantially same formula by the development.

Incidentally, it is preferable that the desired time point t be a time point at which at least the blood flow measuring unit 12 has measured the blood flow volume BF(n). However, the desired time point t may be a time point at which the blood flow measuring unit 12 has not measured the blood flow volume BF(n). In this case, the blood flow volume BF(n) which is measured by the blood flow measuring unit 12 at a time point which is the closest to the desired time point t may be used as the blood flow volume BF(t) at the desired time point t. Alternatively, the blood flow volume BF(t) at the desired time point t which is calculated or estimated from an approximate line or an approximate expression may be used, wherein the approximate line or the approximate expression is obtained by connecting the blood flow volumes BF(n) which are measured by the blood flow measuring unit 12.

More specifically, the explanation using the graph illustrated in FIG. 3 will be provided. A graph at the first step in FIG. 3 illustrates the blood flow volumes BF(n) which are measured by the blood flow measuring unit 12 (incidentally, the approximate line which connects the measured blood flow volumes BF(n) is illustrated for reference). A graph at the second step in FIG. 3 illustrates the blood pressures BPm(n) which are measured by the blood pressure measuring unit 11 (incidentally, the approximate line which connects the measured blood pressures BPm(n) is illustrated for reference). A graph at the third step in FIG. 3 illustrate the blood pressures BPc(n) which are estimated by the blood pressure estimating unit 133 (incidentally, the approximate line which connects the estimated blood pressures BPc(n) is illustrated for reference). Incidentally, FIG. 3 illustrates an example in which the blood pressure BPm(20) which is measured by the blood pressure measuring unit 11 at the time point of “n=20 minutes” is the reference blood pressure BP(s).

As illustrated in FIG. 3, if the blood pressure estimating unit 133 estimates the blood pressure BPc(t) at the desired time point t in the above described aspect, it is understood that the estimated blood pressure BPc(t) varies in an aspect which is same as an aspect in which the blood flow volume BF(n) which is measured by the blood flow measuring unit 12 varies. This is why there is a certain correlation between the blood pressure BP(n) of the living body and the blood flow volume BF(n) of the living body. Therefore, the blood pressure estimating unit 133 appropriately estimates the blood pressure BPc(t) at the desired time point t on the basis of the reference blood pressure BP(s) at the reference time point s, the blood flow volume BF(s) at the reference time point s and the blood flow volume BF(t) at the desired time point t.

Especially, the blood pressure estimating unit 133 estimates the blood pressure BPc(n) on the basis of the blood flow volume BF(n) which is measured at the higher frequency than the blood pressure BPm(n) is. Therefore, even if the blood pressure measuring unit 11 has not measured the blood pressure BPm(t) at the desired time point t, the blood pressure estimating unit 133 is capable of appropriately estimating the blood pressure BPc(t) at the desired time point t. Namely, in the first example, the blood pressure estimating unit 133 is capable of estimating the blood pressure BPc(n) every period which is shorter than a period every which the blood pressure measuring unit 11 measures the blood pressure BPm(n). In other words, the blood pressure estimating unit 133 is capable of estimating the blood pressure BPc(n) at a frequency which is higher than a frequency at which the blood pressure measuring unit 11 measures the blood pressure BPm(n). Moreover, in other words, the blood pressure estimating unit 133 is capable of sequentially estimating the blood pressure BPc(n) on the basis of the blood pressures BPm(n) which are discretely measured by the blood pressure measuring unit 11.

As described above, the blood pressure estimation apparatus 1 in the first example is capable of appropriately estimating the blood pressure BPc(n) of the living body.

### (1-3) First Modified Example

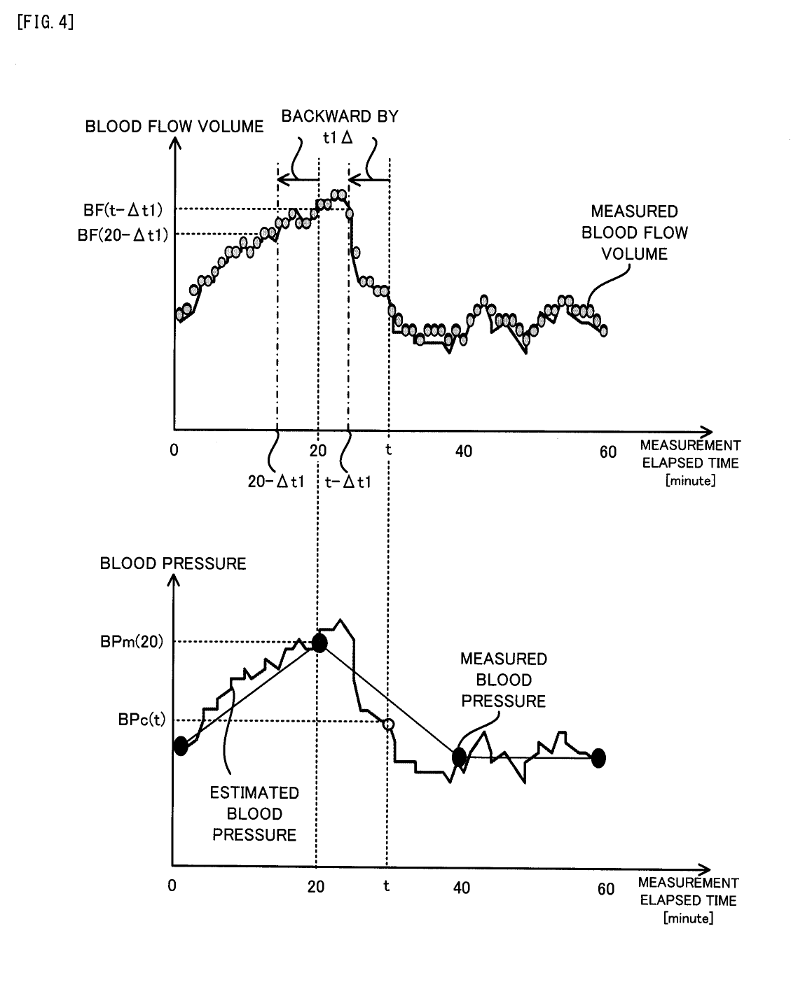

In the above described explanation, the blood pressure estimating unit 133 estimates the blood pressure BPc(t) at the desired time point t on the basis of the blood flow volume BF(s) at the reference time point s and the blood flow volume BF(t) at the desired time point t. However, in a first modified example, the blood pressure estimating unit 133 may estimate the blood pressure BPc(t) at the desired time point t on the basis of the blood flow volume BF(s′) at a time point s′ which is prior to the reference time point s and the blood flow volume BF(t′) at a time point t′ which is prior to at the desired time point t. The first modified example like this will be explained with reference to FIG. 4. FIG. 4 is a graph illustrating the blood pressure BP(n) and the blood flow volume BF(n) which are related to an operation of the blood pressure estimation apparatus 1 which estimates the blood pressure BPc(t) at the desired time point t on the basis of the blood flow volume BF(s′) at the time point s′ which is prior to the reference time point s and the blood flow volume BF(t′) at the time point t′ which is prior to at the desired time point t.

As illustrated in FIG. 4, the blood pressure estimating unit 133 may estimate the blood pressure BPc(t) at the desired time point t on the basis of the blood flow volume BF(s−Δt1) at the time point s−Δt1 which is prior to the reference time point s (in FIG. 4, s=20) by a predetermined timeΔt1 and the blood flow volume BF(t−Δt1) at the time point t−Δt1 which is prior to at the desired time point t by the predetermined time Δt1.

More specifically, the blood pressure estimating unit 133 may regard a value which is obtained by multiplying a ratio A of the blood flow volume BF(t−Δt1) to the blood flow volume BF(s−Δt1) with the reference blood pressure BP(s) at the reference time point s as the blood pressure BPc(t) at the desired time point t. Namely, the blood pressure estimating unit 133 may estimate the blood pressure BPc(t) at the desired time point t by using a formula of BPc(t)=BP(s)*A=BP (s)*(BF(t−Δt1)/BF(s−Δt1)). For example, if the blood pressure BPm(20) which is measured by the blood pressure measuring unit 11 at the time point of “n=20 minutes” is the reference blood pressure BP(s), the blood pressure BPc(t) at the desired time point t is BPm(20)*(BF(t−Δt1)/BF(20-Δt1)).

Alternatively, the blood pressure estimating unit 133 may regard a value which is obtained by adding a multiplied value to the reference blood pressure BP(s) at the reference time point s as the blood pressure BPc(t) at the desired time point t, wherein the multiplied value is obtained by multiplying a change rate B of the blood flow volume BF(t−Δt1) to the blood flow volume BF(s−Δt1) with the reference blood pressure BP(s) at the reference time point s. Namely, the blood pressure estimating unit 133 may estimate the blood pressure BPc(t) at the desired time point t by using a formula of BPc(t)=BP(s)+BP(s)*B=BP (s)+BP(s)*((BF(t−Δt1)−BF(s−Δt1))/BF(s−Δt1)). For example, if the blood pressure BPm(20) which is measured by the blood pressure measuring unit 11 at the time point of “n=20 minutes” is the reference blood pressure BP(s), the blood pressure BPc(t) at the desired time point t is BPm(20)+BPm(20)*((BF(t−Δt1)−BF(20−Δt1)/BF(20−Δt1)).

In this case, for example, if the living body in which a time required for the change of the blood flow volume BF(n) to result in the change of the blood pressure BP(n) is relatively long is a target for the estimation of the blood pressure BPc(n), it is preferable that the blood pressure estimating unit 133 set a relatively large value to the above described Δt1. On the other hand, if the living body in which the time required for the change of the blood flow volume BF(n) to result in the change of the blood pressure BP(n) is relatively short is the target for the estimation of the blood pressure BPc(n), it is preferable that the blood pressure estimating unit 133 set a relatively small value to the above described Δt1. As a result, even if there is an influence of an individual difference of the living body (for example, a variation of a correlation between a temporal change of the blood pressure BP(n) and a temporal change of the blood flow volume BF(n) which is caused by the individual difference), the blood pressure estimating unit 133 is capable of appropriately estimating the blood pressure BPc(t) at the desired time point t while absorbing or compensating the influence of the individual difference.

### (1-4) Second Modified Example

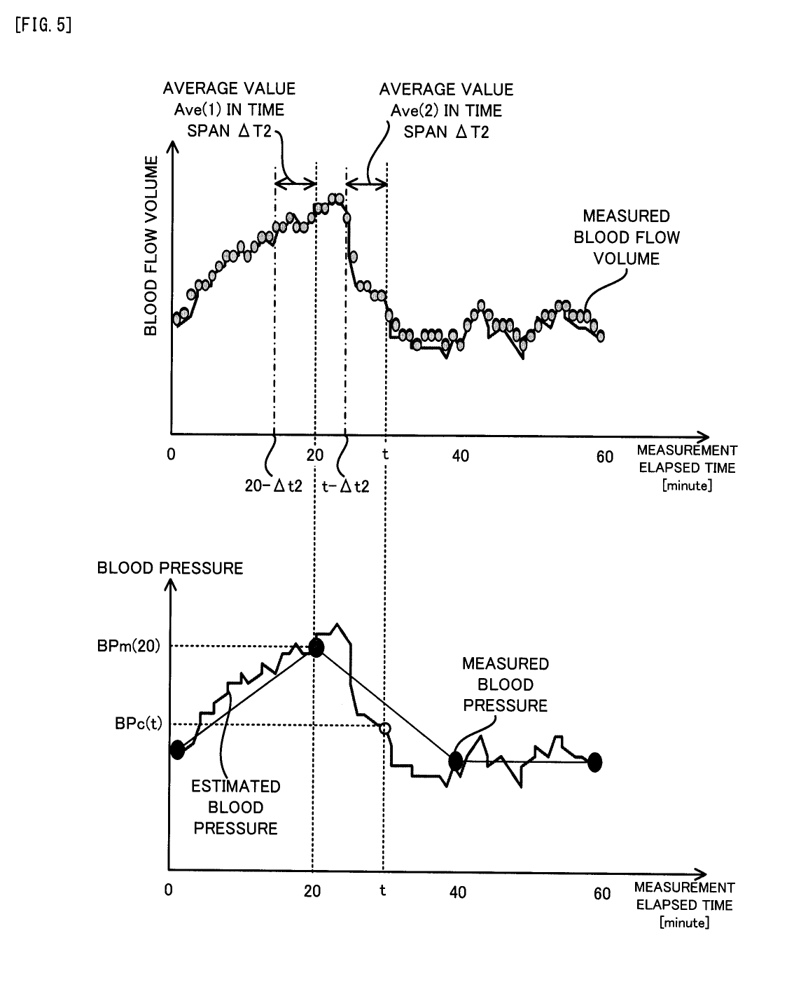

In the above described explanation, the blood pressure estimating unit 133 estimates the blood pressure BPc(t) at the desired time point t on the basis of the blood flow volume BF(s) at the reference time point s and the blood flow volume BF(t) at the desired time point t. However, in a second modified example, the blood pressure estimating unit 133 may estimate the blood pressure BPc(t) at the desired time point t on the basis of an average value of the blood flow volumes BF(n) within a predetermined time span defined by using the reference time point s as a base and an average value of the blood flow volumes BF(n) within a predetermined time span defined by using the desired time point t as a base. The second modified example like this will be explained with reference to FIG. 5. FIG. 5 is a graph illustrating the blood pressure BP(n) and the blood flow volume BF(n) which are related to an operation of the blood pressure estimation apparatus 1 which estimates the blood pressure BPc(t) at the desired time point t on the basis of the average value of the blood flow volumes BF(n) within the predetermined time span defined by using the reference time point s as the base and the average value of the blood flow volumes BF(n) within the predetermined time span defined by using the desired time point t as the base.

As illustrated in FIG. 5, the blood pressure estimating unit 133 may estimate the blood pressure BPc(t) at the desired time point t on the basis of an average value Ave(1) of the blood flow volumes BF(n) within a time span (s−Δt2 to s) which is from the reference time point s (in FIG. 5, s=20) to a time point prior to the reference time point s by Δt2 and an average value Ave(2) of the blood flow volumes BF(n) within a time span (t−Δt2 to t) which is from the desired time point t to a time point prior to the desired time point t by Δt2.

More specifically, the blood pressure estimating unit 133 may regard a value which is obtained by multiplying a ratio A of the average value Ave(2) to the average value Ave(1) with the reference blood pressure BP(s) at the reference time point s as the blood pressure BPc(t) at the desired time point t. Namely, the blood pressure estimating unit 133 may estimate the blood pressure BPc(t) at the desired time point t by using a formula of BPc(t)=BP(s)*A=BP (s)*(Ave(2)/Ave(1)). For example, if the blood pressure BPm(20) which is measured by the blood pressure measuring unit 11 at the time point of “n=20 minutes” is the reference blood pressure BP(s), the blood pressure BPc(t) at the desired time point t is BPm(20)*(Ave(2)/Ave(1)).

Alternatively, the blood pressure estimating unit 133 may regard a value which is obtained by adding a multiplied value to the reference blood pressure BP(s) at the reference time point s as the blood pressure BPc(t) at the desired time point t, wherein the multiplied value is obtained by multiplying a change rate B of the average value Ave(2) to the average value Ave(1) with the reference blood pressure BP(s) at the reference time point s. Namely, the blood pressure estimating unit 133 may estimate the blood pressure BPc(t) at the desired time point t by using a formula of BPc(t)=BP(s)+BP(s)*B=BP (s)+BP(s)*((Ave(2)−Ave(1))/Ave(1)). For example, if the blood pressure BPm(20) which is measured by the blood pressure measuring unit 11 at the time point of “n=20 minutes” is the reference blood pressure BP(s), the blood pressure BPc(t) at the desired time point t is BPm(20)+BPm(20)*((Ave(2)−Ave(1))/Ave(1)).

According to the above described second modified example, even if there is an influence of a pulsation and a fluctuation caused by the living body in the blood flow volume BF(n) which is measured by the blood flow measuring unit 12, the blood pressure estimating unit 133 is capable of appropriately estimating the blood pressure BPc(t) at the desired time point t while absorbing or compensating the influence of the pulsation and the fluctuation caused by the living body.

### (1-5) Third Modified Example

In the above described explanation, the blood pressure estimating unit 133 estimates the blood pressure BPc(t) at the desired time point t on the basis of the ratio A or the change rate B of the blood flow volume BF(t) at the desired time point t to the blood flow volume BF(s) at the reference time point s. However, in a third modified example, the blood pressure estimating unit 133 may perform a weighting process, which depends on a predetermined weighting coefficient α, on the ratio A or the change rate B of the blood flow volume BF(t) at the desired time point t to the blood flow volume BF(s) at the reference time point s. Specifically, the blood pressure estimating unit 133 may multiple the weighting coefficient α with the ratio A or the change rate B of the blood flow volume BF(t) at the desired time point t to the blood flow volume BF(s) at the reference time point s.

More specifically, the blood pressure estimating unit 133 may regard a value which is obtained by multiplying a multiplied value with the reference blood pressure BP(s) at the reference time point s as the blood pressure BPc(t) at the desired time point t, wherein the multiplied value is obtained by multiplying the weighting coefficient α with the ratio A of the blood flow volume BF(t) at the desired time point t to the blood flow volume BF(s) at the reference time point s. Namely, the blood pressure estimating unit 133 may estimate the blood pressure BPc(t) at the desired time point t by using a formula of BPc(t)=BP(s)*(α*A)=BP (s)*α*(BF(t)/BF(s)).

Alternatively, the blood pressure estimating unit 133 may regard a value which is obtained by adding a value, which is obtained by multiplying a multiplied value with the reference blood pressure BP(s) at the reference time point s, to the reference blood pressure BP(s) at the reference time point s as the blood pressure BPc(t) at the desired time point t, wherein the multiplied value is obtained by multiplying the weighting coefficient α with the change rate B of the blood flow volume BF(t) at the desired time point t to the blood flow volume BF(s) at the reference time point s. Namely, the blood pressure estimating unit 133 may estimate the blood pressure BPc(t) at the desired time point t by using a formula of BPc(t)=BP(s)+BP(s)*(α*B)=BP(s)+BP (s)*α*((BF(t)−BF(s))/BF(s)).

Incidentally, if the weighting coefficient α is 1, both formulas are same as the formulas which are used in the above described first example.

In this case, for example, if the living body in which the change of the blood flow volume BF(n) cannot result in the change of the blood pressure BP(n) relatively well is the target for the estimation of the blood pressure BP (n), it is preferable that the blood pressure estimating unit 133 set a relatively small value to the above described weighting coefficient α. On the other hand, if the living body in which the change of the blood flow volume BF(n) can result in the change of the blood pressure BP(n) relatively well is the target for the estimation of the blood pressure BPc(n), it is preferable that the blood pressure estimating unit 133 set a relatively large value to the above described weighting coefficient α. According to the third modified example like this, even if there is the influence of the individual difference of the living body (for example, the variation of the correlation between the temporal change of the blood pressure BP(n) and the temporal change of the blood flow volume BF(n) which is caused by the individual difference), the blood pressure estimating unit 133 is capable of appropriately estimating the blood pressure BPc(t) at the desired time point t while absorbing or compensating the influence of the individual difference.

### (1-6) Fourth Modified Example

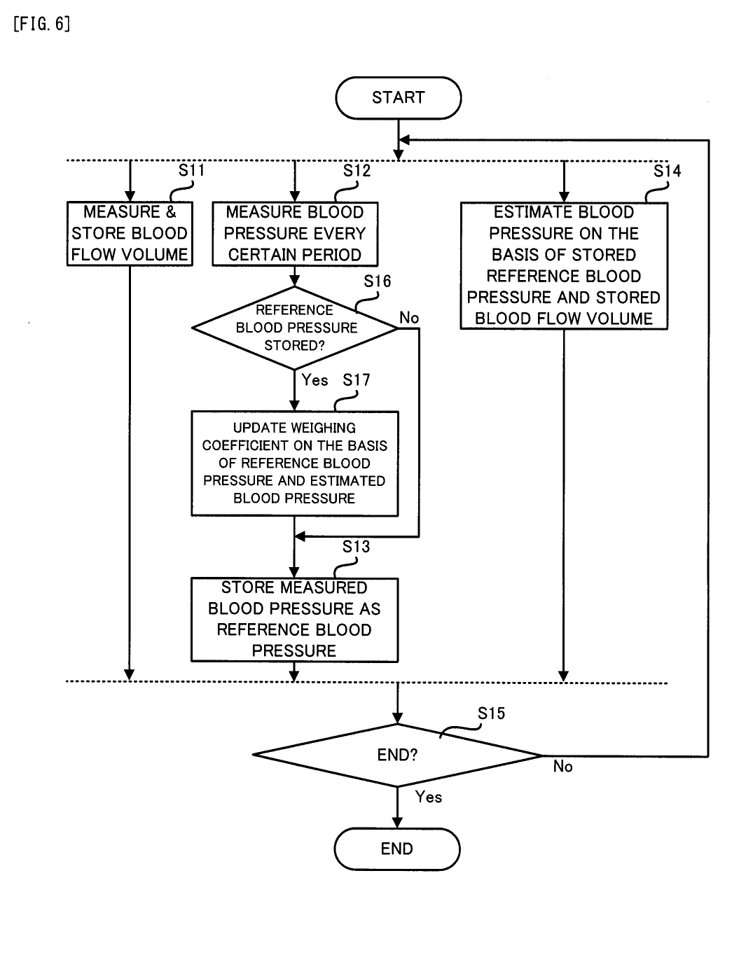

In a fourth modified example, the blood pressure estimating unit 133 may adjust (in other words, change) the above described weighting coefficient α, as occasion demands. Hereinafter, with reference to FIG. 6, an operation of adjusting the weighting coefficient α as occasion demands will be explained. FIG. 6 is a flowchart illustrating the flow of the operation of the blood pressure estimation apparatus 1 in the first example which adjusts a weighting coefficient α as occasion demands. Incidentally, a detailed explanation of an operation which is same as the operation illustrated in FIG. 2 will be omitted by assigning the same step number.

As illustrated in FIG. 6, even in the fourth modified example, the blood flow measuring unit 12 measures the blood flow volume BF(n) of the living body (step S11), as with the first example. Moreover, even in the fourth modified example, subsequent to, in tandem with or in parallel with the measurement of the blood flow volume BF(n) at the step S11, the blood pressure measuring unit 11 measures the blood pressure BPm(n) of the living body (step S12), as with the first example. Moreover, even in the fourth modified example, subsequent to, in tandem with or in parallel with the measurement of the blood flow volume BF(n) at the step S11 and the measurement of the blood pressure BPm(n) at the step S12, the blood pressure estimating unit 133 estimates the blood pressure BPc(n) (step S14), as with the first example.

Especially, in the fourth modified example, the blood pressure measuring unit 11 determines whether or not the reference blood pressure BP(s) is already stored in the reference blood pressure storing unit 131 after measuring the blood pressure BPm(n) at the step S12 (step S16).

As a result of the determination at the step S16, if it is determined that the reference blood pressure BP(s) is stored in the reference blood pressure storing unit 131 (step S16: Yes), the blood pressure estimating unit 133 updates the weighting coefficient α on the basis of the reference blood pressure BPm(s) at the reference time point s and the blood pressure BPc(s) at the reference time point s which is estimated by the blood pressure estimating unit 133 (step S17). Specifically, the blood pressure estimating unit 133 updates the weighting coefficient α such that a difference (especially, an absolute value of the difference) between the reference blood pressure BPm(s) at the new reference time point s which is measured by the blood pressure measuring unit 11 and the blood pressure BPc(s) at the reference time point s which is estimated by the blood pressure estimating unit 13 decreases. Especially, the blood pressure estimating unit 133 may update the weighting coefficient α such that the difference (especially, an absolute value of the difference) between the reference blood pressure BPm(s) at the reference time point s and the blood pressure BPc(s) at the reference time point s which is estimated by the blood pressure estimating unit 13 becomes zero.

On the other hand, as a result of the determination at the step S16, if it is determined that the reference blood pressure BP(s) is not stored in the reference blood pressure storing unit 131 (step S16: No), the blood pressure estimating unit 133 does not need to update the weighting coefficient α.

Then, the reference blood pressure storing unit 131 with which the controller 13 is provided stores the blood pressure BPm(n), which is measured by the blood pressure measuring unit 11 at the step S12, as the reference blood pressure BP(s) (step S13).

According to the above described fourth modified example, the blood pressure estimating unit 133 is capable of the weighting coefficient α such that a false factor of the blood pressure BPc(n) which is actually estimated by the blood pressure estimating unit 133 to the blood pressure BPm(n) which is actually measured by the blood pressure measuring unit 11 becomes small or zero. Therefore, the blood pressure estimating unit 133 is capable of estimating the blood pressure BPc(n) at the desired time point t with higher accuracy by using the updated weighting coefficient α.

Incidentally, the blood pressure estimation apparatus 1 may stores the weighting coefficient α as an archival record every time the measurement of the blood pressure BPm(n), the estimation of the blood pressure BPc(n) or the updating of the weighting coefficient α is performed. For example, the reference blood pressure storing unit 131 which is the memory may stores the weighting coefficient α in addition to the reference blood pressure BP(s). Especially, considering that the weighting coefficient α is an information which is capable of specifying the individual difference of the living body, the weighting coefficient α may be stored in such a manner that the weighting coefficient α can be distinguished for each living body (for example, in a database form including a record in which the weighting coefficient α is associated with a living body ID which is used to uniquely identify the living body). In addition, the weighting coefficient α is adjusted as occasion demands as time goes on. Therefore, the weighting coefficient α may be stored in such a manner that the weighting coefficient α can be distinguished for each time (for example, in a database form including a record in which a series of the weighting coefficients α which has been updated as time goes on is associated with the living body ID which is used to uniquely identify the living body.

If the weighting coefficients α is stored as the archival record, the blood pressure estimating unit 133 may estimate the blood pressure BPc(t) at the desired time t by using the weighting coefficients α which is stored as the archival record. As a result, the blood pressure estimating unit 133 is capable of estimating the blood pressure BPc(t) at the desired time point t with higher accuracy and relatively easily.

### (2) Second Example

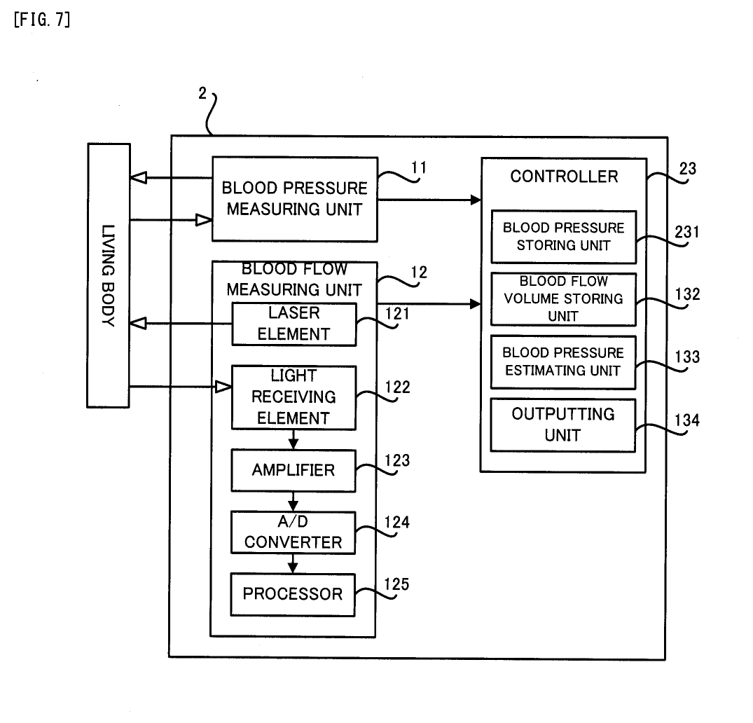

Next, with reference to FIG. 7 to FIG. 10, a blood pressure estimation apparatus 2 in a second example will be explained. Incidentally, a detailed explanation of a configuration and an operation which are respectively same as the configuration and the operation of the blood pressure estimation apparatus 1 in the first example will be omitted by assigning the same reference number and the same step number, respectively.

### (2-1) Configuration of Blood Pressure Estimation Apparatus

Firstly, with reference to FIG. 7, a configuration of the blood pressure estimation apparatus 2 in the second example will be explained. FIG. 7 is a block diagram illustrating the configuration of the blood pressure estimation apparatus 2 in the second example.

As illustrated in FIG. 7, the blood pressure estimation apparatus 2 in the second example is provided with: the blood pressure measuring unit 11; and the blood flow measuring unit 12, as with the blood pressure estimation apparatus 1 in the first example.

The blood pressure estimation apparatus 2 in the second example is further provided with a controller 23. The controller 23 in the second example is different from the controller 13 in the first example in that the controller 23 is provided with a blood pressure storing unit 231 instead of the reference blood pressure storing unit 131. Another component with which the controller 23 in the second example is provided may be same as another component with which the controller 13 in the first example is provided.

The blood pressure storing unit 231 is a memory which stores the blood pressures BPm(n) which are measured by the blood pressure measuring unit 11 and the blood pressures BPc(n) which are estimated by the blood pressure estimating unit 133. Incidentally, in the second example, the blood pressure BPm(n) which is measured by the blood pressure measuring unit 11 is referred to as the “measured blood pressure BPm(n)” and the blood pressure BPc(n) which is estimated by the blood pressure estimating unit 133 is referred to as the “estimated blood pressure BPc(n)”, for the purpose of the explanation. Incidentally, the blood pressure storing unit 231 may store all of the blood pressures BPm(n) which are measured by the blood pressure measuring unit 11, or may selectively store one portion of the blood pressures BPm(n) which are measured by the blood pressure measuring unit 11 (for example, the latest measured blood pressure BPm(n)). Similarly, the blood pressure storing unit 231 may store all of the blood pressures BPc(n) which are estimated by the blood pressure estimating unit 133, or may selectively store one portion of the blood pressures BPc(n) which are estimated by the blood pressure estimating unit 133.

Incidentally, the blood pressure storing unit 231 may be physically separated from the blood flow volume storing unit 132. Alternatively, single memory may constitute the blood pressure storing unit 231 and the blood flow volume storing unit 132.

### (2-2) Operation of Blood Pressure Estimation Apparatus

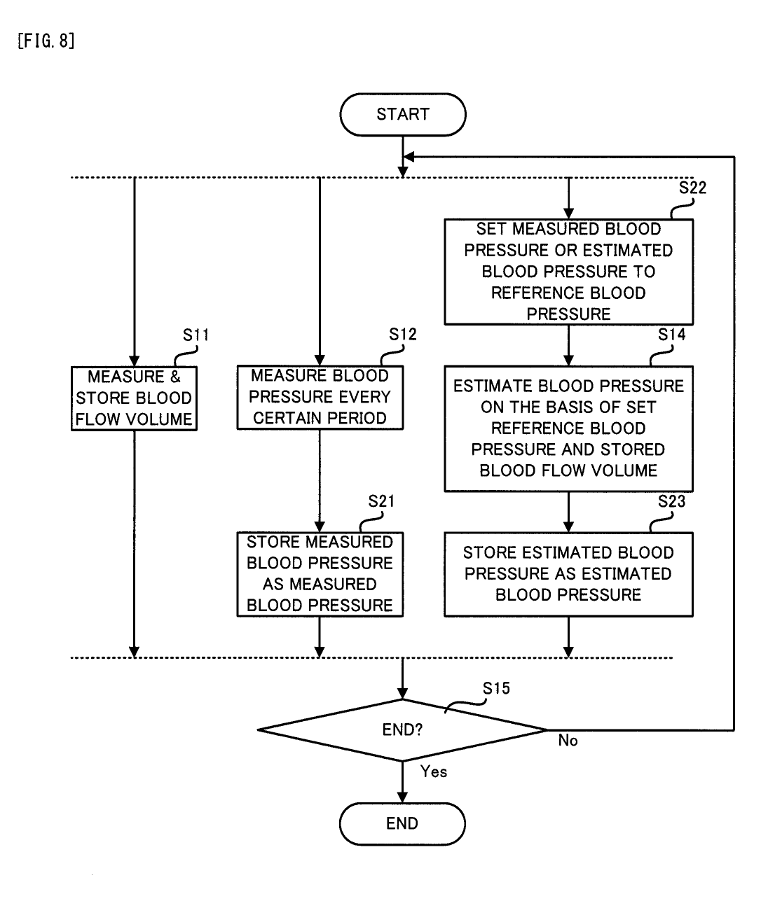

Next, with reference to FIG. 8, a flow of an operation of the blood pressure estimation apparatus 2 in the second example will be explained. FIG. 8 is a flowchart illustrating the flow of the operation of the blood pressure estimation apparatus 2 in the second example.

As illustrated in FIG. 8, even in the second example, the blood flow measuring unit 12 measures the blood flow volume BF(n) of the living body (step S11), as with the first example.

Moreover, even in the second example, subsequent to, in tandem with or in parallel with the measurement of the blood flow volume BF(n) at the step S11, the blood pressure measuring unit 11 measures the blood pressure BPm(n) of the living body (step S12), as with the first example.

Then, the blood pressure BPm(n) which is measured by the blood pressure measuring unit 11 is outputted to the controller 23 (especially, the blood pressure storing unit 231). As a result, the blood pressure storing unit 231 stores the blood pressure BPm(n) which is measured by the blood pressure measuring unit 11 as the measured blood pressure BPm(n) (step S21).

Subsequent to, in tandem with or in parallel with the measurement of the blood flow volume BF(n) at the step S11 and the measurement of the blood pressure BPm(n) at the step S12, the blood pressure estimating unit 133 set either one of the measured blood pressure BPm(n) and the estimated blood pressure BPc(n) which are stored in the blood pressure storing unit 231 to the reference blood pressure BP(s) (step S22). Namely, in the second example, not only the blood pressure BPm(n) which is measured by the blood pressure measuring unit 11 but also the blood pressure BPc(n) which is estimated by the blood pressure estimating unit 133 sometimes becomes the reference blood pressure BP(s).

Then, even in the second example, the blood pressure estimating unit 133 the blood pressure estimating unit 133 estimates the blood pressure BPc(n) (step S14), as with the first example. However, in the second example, the reference blood pressure BP(s) which is used when the blood pressure BPc(n) is estimated is the reference blood pressure BP(s) which is set at the step S22.

Then, the blood pressure BPc(n) which is estimated by the blood pressure estimating unit 133 is outputted to the blood pressure storing unit 231. The blood pressure storing unit 231 stores the blood pressure BPc(n) which is estimated by the blood pressure estimating unit 133 as the estimated blood pressure BPc(n) (step S23).

As described above, the blood pressure estimation apparatus 2 in the second example is capable of appropriately enjoying various effects which the blood pressure estimation apparatus 1 in the first example is capable of enjoying.

In addition, the blood pressure estimation apparatus 2 in the second example is capable of storing the blood pressure BPm(n) which is measured by the blood pressure measuring unit 11 and the blood pressure BPc(n) which is estimated by the blood pressure estimating unit 133. Thus, the blood pressure estimating unit 133 is capable of using, as the reference blood pressure BP(s), not only the blood pressure BPm(n) which is measured by the blood pressure measuring unit 11 but also the blood pressure BPc(n) which is estimated by the blood pressure estimating unit 133.

### (2-3) First Modified Example

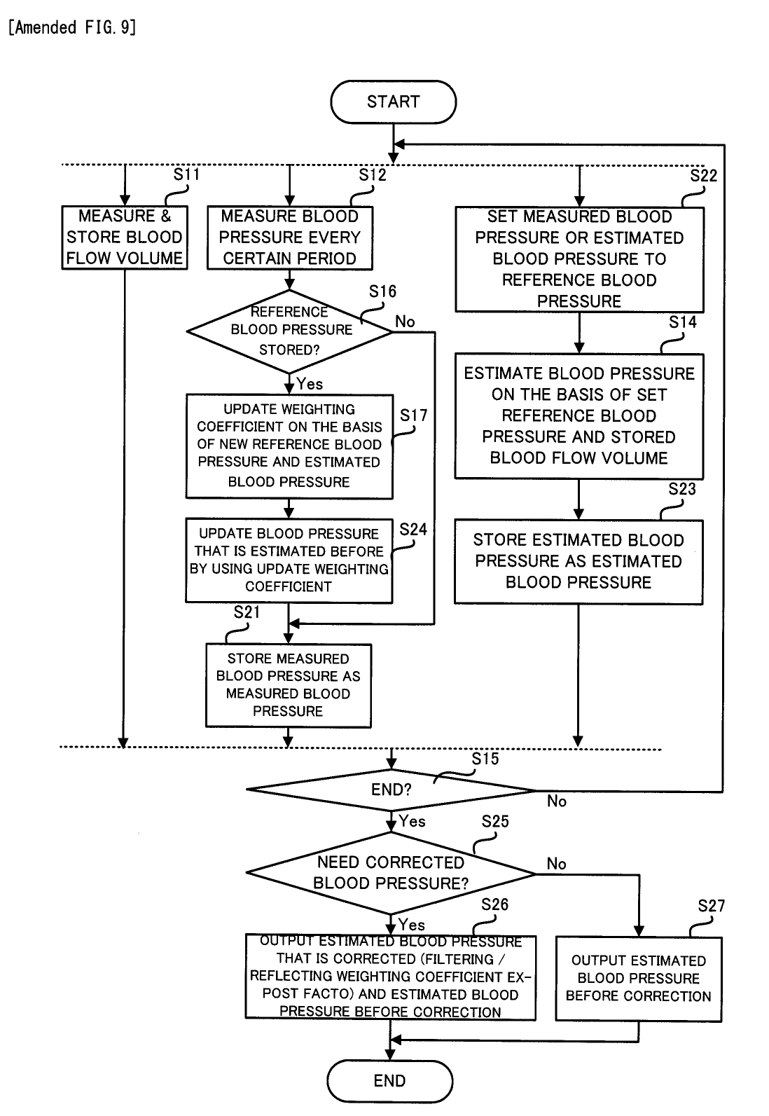

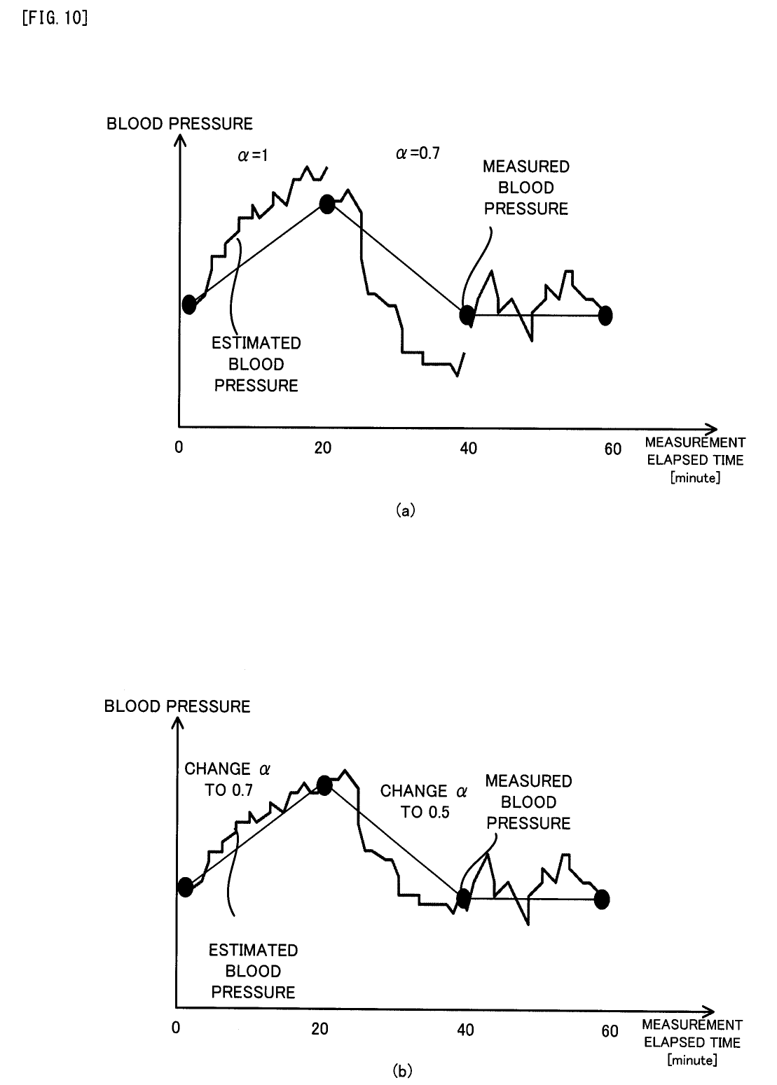

In a first modified example, the blood pressure estimating unit 133 may perform a predetermined filtering process and the like collectively ex-post facto on the estimated blood pressures BPc(n) within a certain time span after estimating the blood pressures BPc(n) within the certain time span, by using the fact that the estimated blood pressures BPc(n) which is estimated by the blood pressure estimating unit 133 are stored in the blood pressure storing unit 231. Alternatively, if the weighting coefficient α which is explained in the third modified example of the first example is used, the blood pressure estimating unit 133 may reflect the weighting coefficient α correctively ex-post facto on the estimated blood pressures BPc(n) within the certain time span after estimating the blood pressures BPc(n) within the certain time span. An operation in this case will be explained with reference to FIG. 9. FIG. 9 is a flowchart illustrating a flow of the first modified example of the operation of the blood pressure estimation apparatus 2 in the second example. FIG. 10 is a graph illustrating the blood pressure and the blood flow volume which are related to the first modified example of the operation of the blood pressure estimation apparatus 2 in the second example.

As illustrated in FIG. 9, even in the first modified example, the blood flow measuring unit 12 measures the blood flow volume BF(n) of the living body (step S11), as with the second example.

Moreover, even in the first modified example, subsequent to, in tandem with or in parallel with the measurement of the blood flow volume BF(n) at the step S11, the blood pressure measuring unit 11 measures the blood pressure BPm(n) of the living body (step S12), as with the second example.

Moreover, even in the first modified example, subsequent to, in tandem with or in parallel with the measurement of the blood flow volume BF(n) at the step S11 and the measurement of the blood pressure BPm(n) at the step S12, the blood pressure estimating unit 133 set either one of the measured blood pressure BPm(n) and the estimated blood pressure BPc(n) which are stored in the blood pressure storing unit 231 to the reference blood pressure BP(s) (step S22). Then, the blood pressure estimating unit 133 estimates the blood pressure BPc(n) (step S14). Then, the blood pressure storing unit 231 stores the blood pressure BPc(n) which is estimated by the blood pressure estimating unit 133 as the estimated blood pressure BPc(n) (step S23).

Even in the first modified example, the blood pressure measuring unit 11 determines whether or not the reference blood pressure BP(s) is already stored in the blood pressure storing unit 231 after measuring the blood pressure BPm(n) at the step S12 (step S16), as with the second example.

As a result of the determination at the step S16, if it is determined that the reference blood pressure BP(s) is stored in the blood pressure storing unit 231 (step S16: Yes), the blood pressure estimating unit 133 updates the weighting coefficient α on the basis of the reference blood pressure BPm(s) at the reference time point s which is measured by the blood pressure measuring unit 11 and the blood pressure BPc(s) at the reference time point s which is estimated by the blood pressure estimating unit 133 (step S17).

Especially, in the first modified example, the blood pressure estimating unit 133 updates the blood pressure BPc(n) which has been already estimated before by using the updated weighting coefficient α (step S24). In other words, the blood pressure estimating unit 133 updates the estimated blood pressure BPc(n) which is stored in the blood pressure storing unit 231 by using the updated weighting coefficient α. For example, the blood pressure estimating unit 133 replaces the estimated blood pressure BPc(n) which is estimated by using the weighting coefficient α before the updating by the estimated blood pressure BPc(n) which is estimated by using the weighting coefficient α after the updating.

On the other hand, As a result of the determination at the step S16, if it is determined that the reference blood pressure BP(s) is not stored in the blood pressure storing unit 231 (step S16: No), the blood pressure estimating unit 133 does not need to update the weighting coefficient α. In addition, the blood pressure does not need to update the blood pressure BPc(n) which has been already estimated before.

Then, the blood pressure storing unit 231 stores the blood pressure BPm(n) which is measured by the blood pressure measuring unit 11 as the measured blood pressure BPm(n) (step S21).

Furthermore in the first modified example, if it is determined that the operation of estimating the estimated blood pressure BPc(n) by the blood pressure estimation apparatus 2 is ended (step S15: No), the outputting unit 134 determines whether or not to output the estimated blood pressure BPc(n) which is corrected (step S25). For example, the outputting unit 134 may determine whether or not to output the estimated blood pressure BPc(n) which is corrected by monitoring an instruction or a requirement from a user.

As a result of the determination at the step S25, if it is determined that the blood pressure BP(n) which is corrected needs to be outputted (step S25: Yes), the blood pressure estimating unit 133 performs a predetermined correction process on the blood pressure BP(n) which has been estimated ever (step S26). In other words, the blood pressure estimating unit 133 performs the predetermined correction process on the estimated blood pressure BPc(n) which is stored in the blood pressure storing unit 231 (step S26).

Specifically, the blood pressure estimating unit 133 may update the weighting coefficient α ex-post facto again, on the basis of the estimated blood pressure BPc(n) and the measured blood pressure BPm(n) which are stored in the blood pressure storing unit 231. Then, the blood pressure estimating unit 133 may update the estimated blood pressure BPc(n) which is stored in the blood pressure storing unit 231 by using the weighting coefficient α which is updated ex-post facto.

Alternatively, the blood pressure estimating unit 133 may perform the predetermined filtering process on the estimated blood pressure BPc(n) which are stored in the blood pressure storing unit 231. As one example of the predetermined filtering process, a process of converting the estimated blood pressure BPc(n) in a more visible format, a process of reducing or eliminating an influence of the estimated blood pressure BPc(n) which is possibly estimated abnormally, a process of reducing or eliminating an arbitrary noise (for example, a noise of a predetermined frequency) and the like are listed. For example, the blood pressure estimating unit 133 may update the estimated blood pressure BPc(t) at the desired time point t by using a formula of BPc(t)=B1*BPc(t−1)+B2*BPc(t−2)+ . . . +Bk*BPc(t−k) (incidentally, B1 to Bk are arbitrary constant number).

On the other hand, as a result of the determination at the step S25, if it is determined that the blood pressure BP(n) which is corrected does not need to be outputted (step S25: No), the outputting unit 134 outputs the estimated blood pressure BPc(n) which is estimated by the blood pressure estimating unit 133 without change. In other words, the outputting unit 134 outputs the estimated blood pressure BPc(n) which is stored in the blood pressure storing unit 231 without change.

### (3) Third Example

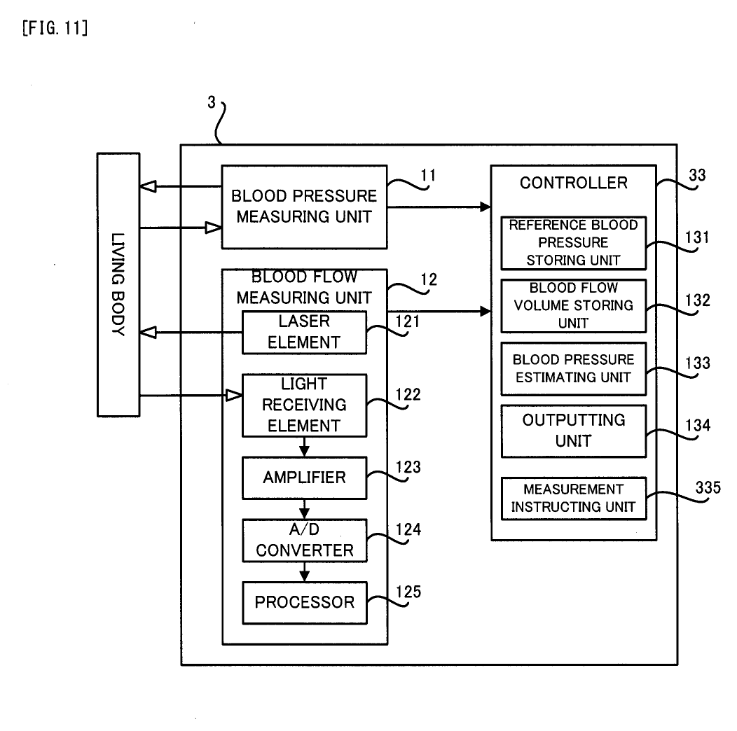

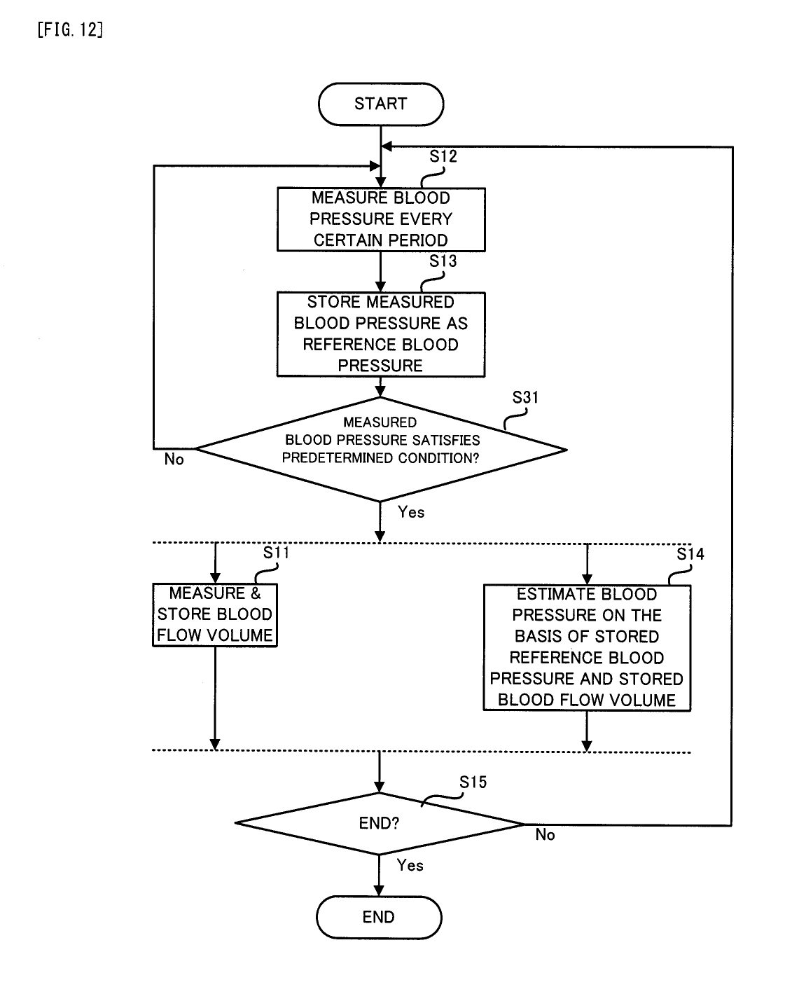

Next, with reference to FIG. 11 to FIG. 12, a blood pressure estimation apparatus 3 in a third example will be explained. Incidentally, a detailed explanation of a configuration and an operation which are respectively same as the configuration and the operation of the blood pressure estimation apparatus 1 in the first example to the blood pressure estimation apparatus 2 in the second example will be omitted by assigning the same reference number and the same step number, respectively.

### (3-1) Configuration of Blood Pressure Estimation Apparatus

Firstly, with reference to FIG. 11, a configuration of the blood pressure estimation apparatus 3 in the third example will be explained. FIG. 11 is a block diagram illustrating the configuration of the blood pressure estimation apparatus 3 in the third example.

As illustrated in FIG. 11, the blood pressure estimation apparatus 3 in the third example is provided with: the blood pressure measuring unit 11; and the blood flow measuring unit 12, as with the blood pressure estimation apparatus 1 in the first example.

The blood pressure estimation apparatus 3 in the third example is further provided with a controller 33. The controller 33 in the third example is different from the controller 13 in the first example in that the controller 33 is further provided with a measurement instructing unit 335. Another component with which the controller 33 in the third example is provided may be same as another component with which the controller 12 in the first example is provided.

The measurement instructing unit 335 may control the blood flow measuring unit 12 such that the blood flow measuring unit 12 measures the blood flow volume BF(n) when the blood pressure BPm(n) which is measured by the blood pressure measuring unit 11 satisfies a predetermined condition. In other words, the measurement instructing unit 335 may control the blood flow measuring unit 12 such that the blood flow measuring unit 12 does not measure the blood flow volume BF(n) when the blood pressure BPm(n) which is measured by the blood pressure measuring unit 11 does not satisfy the predetermined condition.

The measurement instructing unit 335 may control the blood pressure estimating unit 133 such that the blood pressure estimating unit 133 estimates the blood pressure BPc(n) when the blood pressure BPm(n) which is measured by the blood pressure measuring unit 11 satisfies the predetermined condition, in addition to or instead of controlling the blood flow measuring unit 12. In other words, the measurement instructing unit 335 may control the blood pressure estimating unit 133 such that the blood pressure estimating unit 133 does not estimate the blood pressure BPc(n) when the blood pressure BPm(n) which is measured by the blood pressure measuring unit 11 does not satisfy the predetermined condition.

### (2-2) Operation of Blood Pressure Estimation Apparatus

Next, with reference to FIG. 12, a flow of an operation of the blood pressure estimation apparatus 3 in the third example will be explained. FIG. 12 is a flowchart illustrating the flow of the operation of the blood pressure estimation apparatus 3 in the third example.

As illustrated in FIG. 12, even in the third example, the blood pressure measuring unit 11 measures the blood pressure BPm(n) of the living body (step S12), as with the first example. Moreover, the reference blood pressure storing unit 131 stores the blood pressure BPm(n) which is measured by the blood pressure measuring unit 11 as the reference blood pressure BP (s) (step S13).

Especially in the third example, the measurement instructing unit 335 determines whether or not the blood pressure BPm(n) which is measured at the step S12 satisfies the predetermined condition (step S31). For example, the measurement instructing unit 335 may determine whether or not the blood pressure BPm(n) which is measured at the step S12 is a value which needs to be monitored continuously (for example, a value which is not a normal value, a value which is a normal value but requires the attention, and the like). If the blood pressure BPm(n) which is measured at the step S12 is the value which needs to be monitored, it may be determined that the blood pressure BPm(n) which is measured at the step S12 satisfies the predetermined condition. On the other hand, if the blood pressure BPm(n) which is measured at the step S12 is not the value which needs to be monitored, it may be determined that the blood pressure BPm(n) which is measured at the step S12 does not satisfy the predetermined condition. Alternatively, the measurement instructing unit 335 may determine that the blood pressure BPm(n) which is measured at the step S12 satisfies the predetermined condition by determining whether or not the blood pressure BPm(n) which is measured at the step S12 is another desired value or predetermined value or is within a predetermined range.

As a result of the determination at the step S31, if it is determined that the blood pressure BPm(n) which is measured at the step S12 does not satisfy the predetermined condition (step S31: No), the operations at the step S12, the step S13 and the step S31 are repeated. Namely, the blood pressure measuring unit 11 measures the blood pressure BPm(n) of the living body. The reference blood pressure storing unit 131 stores the blood pressure BPm(n) which is measured by the blood pressure measuring unit 11 as the reference blood pressure BP(s). The measurement instructing unit 335 determines whether or not the blood pressure BPm(n) which is measured at the step S12 satisfies the predetermined condition.

In addition, in this case, the measurement instructing unit 335 may control the blood flow measuring unit 12 such that the blood flow measuring unit 12 does not measure the blood flow volume BF(n). As a result, the blood flow measuring unit 12 does not need to measure the blood flow volume BF(n). Moreover, the measurement instructing unit 335 may control the blood pressure estimating unit 133 such that the blood pressure estimating unit 133 does not estimate the blood pressure BPc(n), in addition to or instead of controlling the blood flow measuring unit 12. As a result, the blood pressure estimating unit 133 does not need to estimate the blood pressure BPc(n).

On the other hand, as a result of the determination at the step S31, if it is determined that the blood pressure BPm(n) which is measured at the step S12 satisfies the predetermined condition (step S31: Yes), the measurement instructing unit 335 may control the blood flow measuring unit 12 such that the blood flow measuring unit 12 measures the blood flow volume BF(n). As a result, the blood flow measuring unit 12 measures the blood flow volume BF(n) (step S11). Moreover, the measurement instructing unit 335 may control the blood pressure estimating unit 133 such that the blood pressure estimating unit 133 estimates the blood pressure BPc(n), in addition to or instead of controlling the blood flow measuring unit 12. As a result, the blood pressure estimating unit 133 estimates the blood pressure BPc(n) (step S14).

In addition, even if it is determined that the blood pressure BPm(n) which is measured at the step S12 satisfies the predetermined condition, it is preferable that the blood pressure measuring unit 11 measure the blood pressure BPm(n) of the living body. If the blood pressure measuring unit 11 measures the blood pressure BPm(n), it is preferable that the measurement instructing unit 335 determine whether or not the newly measured blood pressure BPm(n) satisfies the predetermined condition.

As described above, the blood pressure estimation apparatus 3 in the third example is capable of appropriately enjoying various effects which the blood pressure estimation apparatus 1 in the first example is capable of enjoying.

In addition, the blood pressure estimation apparatus 3 in the third example performs the measurement of the blood flow volume BF(n) and the estimation of the blood pressure BPc(n) when the blood pressure BPm(n) which is measured by the blood pressure measuring unit 11 satisfies the predetermined condition. In other words, the blood pressure estimation apparatus 3 in the third example does not need to perform the measurement of the blood flow volume BF(n) and the estimation of the blood pressure BPc(n) when the blood pressure BPm(n) which is measured by the blood pressure measuring unit 11 does not satisfy the predetermined condition. Thus, a power consumption of the blood pressure estimation apparatus 3 decreases, in comparison with a blood pressure estimation apparatus in a comparative example which always keeps performing the measurement of the blood flow volume BF(n) and the estimation of the blood pressure BPc(n).

### (4) Fourth Example

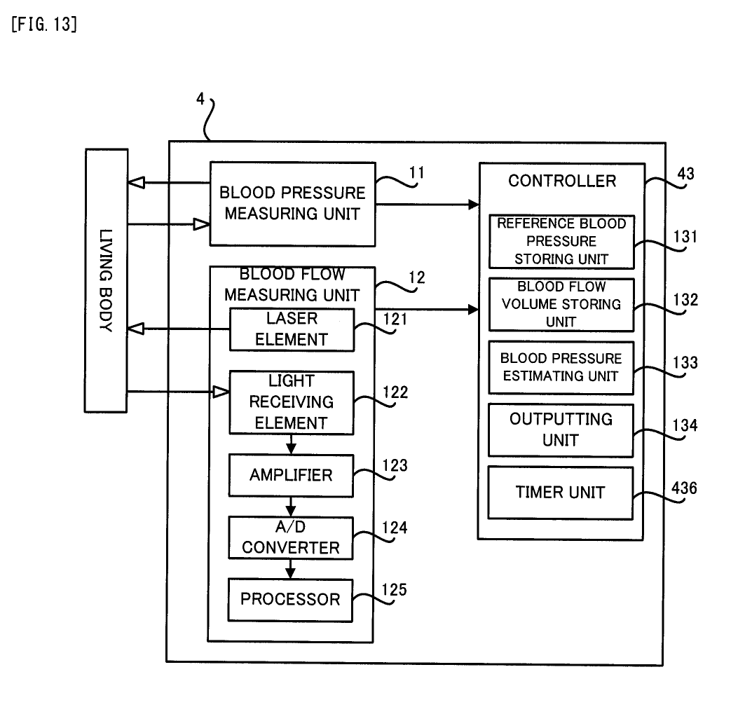

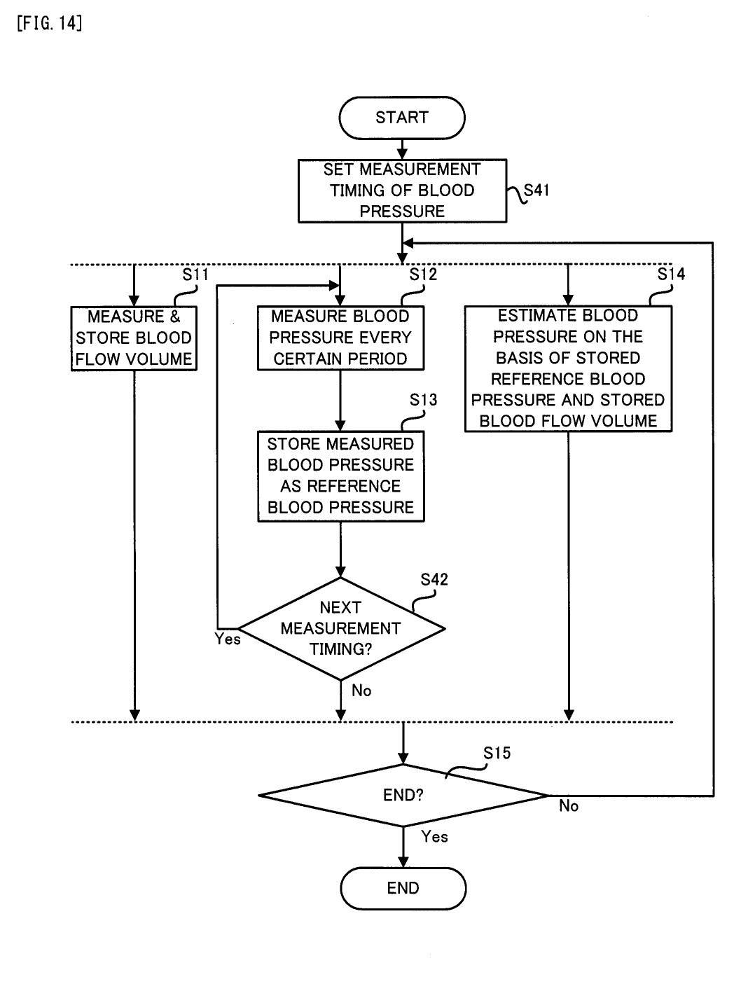

Next, with reference to FIG. 13 to FIG. 14, a blood pressure estimation apparatus 4 in a fourth example will be explained. Incidentally, a detailed explanation of a configuration and an operation which are respectively same as the configuration and the operation of the blood pressure estimation apparatus 1 in the first example to the blood pressure estimation apparatus 3 in the third example will be omitted by assigning the same reference number and the same step number, respectively.

### (4-1) Configuration of Blood Pressure Estimation Apparatus

Firstly, with reference to FIG. 13, a configuration of the blood pressure estimation apparatus 4 in the fourth example will be explained. FIG. 13 is a block diagram illustrating the configuration of the blood pressure estimation apparatus 4 in the fourth example.

As illustrated in FIG. 13, the blood pressure estimation apparatus 4 in the fourth example is provided with: the blood pressure measuring unit 11; and the blood flow measuring unit 12, as with the blood pressure estimation apparatus 1 in the first example.

The blood pressure estimation apparatus 4 in the fourth example is further provided with a controller 43. The controller 43 in the fourth example is different from the controller 13 in the first example in that the controller 43 is further provided with a timer unit 436. Another component with which the controller 43 in the fourth example is provided may be same as another component with which the controller 13 in the first example is provided.

The timer unit 436 sets a timing at which the blood pressure measuring unit 11 measures the blood pressure BPm(n). In addition, the timer unit 436 controls the blood pressure measuring unit 11 to measure the blood pressure BPm(n) when the timing at which the blood pressure measuring unit 11 measures the blood pressure BPm(n) comes.

### (4-2) Operation of Blood Pressure Estimation Apparatus

Next, with reference to FIG. 14, a flow of an operation of the blood pressure estimation apparatus 4 in the fourth example will be explained. FIG. 14 is a flowchart illustrating the flow of the operation of the blood pressure estimation apparatus 4 in the fourth example.

As illustrated in FIG. 14, the timer unit 436 sets the timing at which the blood pressure measuring unit 11 measures the blood pressure BPm(n) (step S41). For example, the timer unit 436 may set a period (for example, a period of 20 minutes) as the timing at which the blood pressure measuring unit 11 measures the blood pressure BPm(n). The timer unit 436 may set a time point itself (for example, a time point of 20 minutes, 40 minutes, 60 minutes, . . . ) as the timing at which the blood pressure measuring unit 11 measures the blood pressure BPm(n).

Then, even in the fourth example, the blood flow measuring unit 12 measures the blood flow volume BF(n) of the living body (step S11), as with the first example. In addition, even in the fourth example, the blood pressure estimating unit 133 the blood pressure estimating unit 133 estimates the blood pressure BPc(n) (step S14), as with the first example.

Moreover, even in the fourth example, subsequent to, in tandem with or in parallel with the measurement the measurement of the blood flow volume BF(n) at the step S11, the blood pressure measuring unit 11 measures the blood pressure BPm(n) of the living body (step S12), as with the first example. In addition, the reference blood pressure storing unit 131 stores the blood pressure BPm(n) which is measured by the blood pressure measuring unit 11 as the reference blood pressure BP(s) (step S13).

Then, in the fourth example, the timer unit 436 determines whether or not a current timing is the timing which is set at the step S41 (namely, the timing at which the blood pressure measuring unit 11 measures the blood pressure BPm(n)) (step S42). In other words, the timer unit 436 determines whether or not the timing which is set at the step S41 (namely, the timing at which the blood pressure measuring unit 11 measures the blood pressure BPm(n)) comes (step S42).

As a result of the determination at the step S42, if it is determined that the current timing is the timing which is set at the step S41 (namely, the timing at which the blood pressure measuring unit 11 measures the blood pressure BPm(n)) (step S42: Yes), the operations at the step S12 and the step S13 are repeated. Namely, the blood pressure measuring unit 11 measures the blood pressure BPm(n) of the living body (step S12). In addition, the reference blood pressure storing unit 131 stores the blood pressure BPm(n) which is measured by the blood pressure measuring unit 11 as the reference blood pressure BP(s) (step S13).

On the other hand, as a result of the determination at the step S42, if it is determined that the current timing is not the timing which is set at the step S41 (namely, the timing at which the blood pressure measuring unit 11 measures the blood pressure BPm(n)) (step S42: No), the operations at the step S12 and the step S13 do not need to be performed. Namely, the blood pressure measuring unit 11 does not need to measure the blood pressure BPm(n) of the living body until the timing which is set at the step S41 (namely, the timing at which the blood pressure measuring unit 11 measures the blood pressure BPm(n)) comes again. In addition, the reference blood pressure storing unit 131 does not need to store the blood pressure BPm(n) which is measured by the blood pressure measuring unit 11 as the reference blood pressure BP(s) until the timing which is set at the step S41 (namely, the timing at which the blood pressure measuring unit 11 measures the blood pressure BPm(n)) comes again.

As described above, the blood pressure estimation apparatus 4 in the fourth example is capable of appropriately enjoying various effects which the blood pressure estimation apparatus 1 in the first example is capable of enjoying.

In addition, the blood pressure estimation apparatus 4 in the fourth example is capable of automatically measuring the blood pressure BPm(n) at a desired timing by the operation of the timer unit 436. Thus, the blood pressure estimation apparatus 4 in the fourth example is capable of automatically measuring the blood pressure BPm(n) even when the operator does not match the timing (alternatively, the operator does not perform the manual operation).

Incidentally, the timer unit 436 may change the set timing depending on whether or not the blood pressure BPm(n) which is measured by the blood pressure measuring unit 11 satisfies the predetermined condition. For example, the timer unit 436 may set a relatively short period as the timing at which the blood pressure measuring unit 11 measures the blood pressure BPm(n), if the blood pressure BPm(n) which is measured by the blood pressure measuring unit 11 satisfies the predetermined condition (for example, is the value which needs to be monitored continuously). As a result, the blood pressure measuring unit 11 measures the blood pressure BPm(n) at a relatively high frequency, if the blood pressure BPm(n) which is measured by the blood pressure measuring unit 11 satisfies the predetermined condition. On the other hand, the timer unit 436 may set a relatively long period as the timing at which the blood pressure measuring unit 11 measures the blood pressure BPm(n), if the blood pressure BPm(n) which is measured by the blood pressure measuring unit 11 does not satisfy the predetermined condition (for example, is not the value which needs to be monitored continuously). As a result, the blood pressure measuring unit 11 measures the blood pressure BPm(n) at a relatively low frequency, if the blood pressure BPm(n) which is measured by the blood pressure measuring unit 11 does not satisfy the predetermined condition. Thus, the blood pressure measuring unit 11 is capable of measuring the blood pressure BPm(n) at an appropriate frequency which depends on whether or not the blood pressure BPm(n) is the value which needs to be monitored continuously.

Incidentally, one part of the components which are explained in the first example to the fourth example can be combined as occasion demand. Even in this case, the blood flow volume detection apparatus, which is obtained by combining one part of the components which are explained in the first example to the fourth example, is capable of enjoying the above described various effects.

The present invention can be changed without departing from the essence or spirit of the invention which can be read from the claims and the entire specification. A blood pressure estimation apparatus and method, which involve such changes, is also intended to be within the technical scope of the present invention.

### DESCRIPTION OF REFERENCE SIGNS

1, 2, 3, 4 blood pressure estimation apparatus
11 blood pressure measuring unit
12 blood flow measuring unit
121 laser element
122 light receiving element
123 amplifier
124 A/D converter
125 processor
13, 23, 33, 43 controller
131 reference blood pressure storing unit
132 blood flow volume storing unit
133 blood pressure estimating unit
134 outputting unit
231 blood pressure storing unit
335 measurement instructing unit
436 timer unit

1. A blood pressure estimation apparatus comprising:
a blood pressure measuring device which measures a blood pressure of a living body every first period;
a blood flow measuring device which measures a blood flow volume of the living body every second period which is shorter than the first periods; and
a blood pressure estimating device which estimates the blood pressure every third period which is shorter than the first periods, on the basis of the blood pressure which is measured by the blood pressure measuring device and the blood flow volume which is measured by the blood flow measuring device, wherein
the blood pressure estimating device estimates the blood pressure at a desired time point on the basis of the blood pressure at a reference time point which is measured by the blood pressure measuring device and a ratio or a change rate of the blood flow volume at the desired time point which is measured by the blood flow measuring device to the blood flow volume at the reference time point which is measured by the blood flow measuring device,
the ratio or the change rate is weighted depending on a predetermined reflecting coefficient.

2-3. (canceled)

4. The blood pressure estimation apparatus according to claim 1 further comprising: a storing device which stores the blood pressure which is estimated by the blood pressure estimating device within a predetermined estimating time span, wherein
the blood pressure estimating device estimates the blood pressure within the estimating time span, and then collectively corrects the blood pressure within the estimating time span ex-post facto by collectively weighing the blood pressure which is stored in the storing device and which is estimated within the estimating time span on the basis of the reflecting coefficient.

5. The blood pressure estimation apparatus according to claim 4, wherein
the blood pressure estimating device corrects the reflecting coefficient such that a difference between the blood pressure at the reference time point which is measured by the blood pressure measuring device and the blood pressure at the reference time point which is estimated by the blood pressure estimating device decreases.

6. The blood pressure estimation apparatus according to claim 5 further comprising: a storing device which stores the reflecting coefficient which is corrected by the blood pressure estimating device, wherein
the blood pressure estimating device estimates the blood pressure at the desired time point on the basis of the ratio or the change rate which is weighted depending on the reflecting coefficient which is stored in the storing device.

7. The blood pressure estimation apparatus according to claim 1 further comprising: a storing device which stores the blood pressure which is estimated by the blood pressure estimating device within a predetermined estimating time span, wherein
the blood pressure estimating device estimates the blood pressure within the estimating time span, and then collectively corrects the blood pressure within the estimating time span ex-post facto by collectively performing a predetermined filtering process on the blood pressure which is stored in the storing device and which is estimated within the estimating time span.

8. The blood pressure estimation apparatus according to claim 1, wherein
the blood pressure estimating device estimates the blood pressure at a desired time point on the basis of the blood pressure at a reference time point which is measured by the blood pressure measuring device and a ratio or a change rate of the blood flow volume at a time point prior to the desired time point by a predetermined time which is measured by the blood flow measuring device to the blood flow volume at a time point prior to the reference time point by a predetermined time which is measured by the blood flow measuring device.

9. The blood pressure estimation apparatus according to claim 1, wherein
the blood pressure estimating device estimates the blood pressure at a desired time point on the basis of the blood pressure at a reference time point which is measured by the blood pressure measuring device and a ratio or a change rate of an average value of the blood flow volume within a predetermined time span defined by using the desired time point as a base which is measured by the blood flow measuring device to an average value of the blood flow volume within a predetermined time span defined by using the reference time point as a base which is measured by the blood flow measuring device.

10. The blood pressure estimation apparatus according to claim 1, wherein
the blood pressure estimating device (i) estimates the blood pressure if the blood pressure which is measured by the blood pressure measuring device satisfies a predetermined condition, and (ii) does not estimate the blood pressure if the blood pressure which is measured by the blood pressure measuring device does not satisfy the predetermined condition.

11. The blood pressure estimation apparatus according to claim 1, wherein
the blood flow measuring device (i) measures the blood flow volume if the blood pressure which is measured by the blood pressure measuring device satisfies a predetermined condition, and (ii) does not measure the blood flow volume if the blood pressure which is measured by the blood pressure measuring device does not satisfy the predetermined condition.

12. The blood pressure estimation apparatus according to claim 1 further comprising: a timer device which sets a timing at which the blood pressure measuring device measures the blood pressure, wherein
the blood pressure measuring device automatically measures the blood pressure in the first periods by measuring blood pressure at the timing which is set by the timer device.

13. The blood pressure estimation apparatus according to claim 12, wherein
the timer device sets the timing such that (i) a frequency of the timing at which the blood pressure is measured when the blood pressure which is measured by the blood pressure measuring device satisfies a predetermined condition is higher than (ii) a frequency of the timing at which the blood pressure is measured when the blood pressure which is measured by the blood pressure measuring device does not satisfy the predetermined condition.

14. A blood pressure estimation method comprising:
a blood pressure measuring process which measures a blood pressure of a living body every first period;
a blood flow measuring process which measures a blood flow volume of the living body every second period which is shorter than the first period; and
a blood pressure estimating process which estimates the blood pressure every third period which is shorter than the first period, on the basis of the blood pressure which is measured by the blood pressure measuring process and the blood flow volume which is measured by the blood flow measuring process, wherein
the blood pressure estimating process estimates the blood pressure at a desired time point on the basis of the blood pressure at a reference time point which is measured by the blood pressure measuring process and a ratio or a change rate of the blood flow volume at the desired time point which is measured by the blood flow measuring process to the blood flow volume at the reference time point which is measured by the blood flow measuring process,
the ratio or the change rate is weighted depending on a predetermined reflecting coefficient.
